(19) United States
(12) Patent Application Publication
(10) Pub. No.: US 2014/0018435 A1
(43) Pub. Date: Jan. 16, 2014

(54) Transdermal Delivery of Therapeutic Agents Using Poly (Amidoamine) Dendrimers

(71) Applicant: UNIVERSITY OF ILLINOIS CHICAGO, Chicago, IL (US)

(72) Inventors: Seungpyo Hong, Naperville, IL (US); Robert Chatterton, Chicago, IL (US); Seema Khan, Chicago, IL (US); Oukseub Lee, Morton Grove, IL (US); Yang Yang, Chicago, IL (US)

(21) Appl. No.: 13/906,009

(22) Filed: May 30, 2013

Related U.S. Application Data
(60) Provisional application No. 61/656,335, filed on Jun. 6, 2012.

(51) Int. Cl. A61K 47/48; A61K 31/138

(52) U.S. Cl. CPC A61K 47/48207; A61K 31/138
USPC 514/648

(57) ABSTRACT

The invention provides for compositions for transdermal delivery of a therapeutic agent associated with a surface modified poly(amidoamine) PAMAM dendrimer, wherein the surface modified dendrimer increased skin penetration of the therapeutic agent. The invention particularly provides for compositions and methods for transdermal delivery of anticancer and chemo-preventive agents.

This application claims priority to U.S. Provisional Application No. 61/656,335, filed on Jun. 6, 2012, which is incorporated by reference in its entirety.

### FIELD OF INVENTION

The invention provides for compositions for transdermal delivery of a therapeutic agent associated with a surface modified poly(amidoamine) PAMAM dendrimer, wherein the surface modified dendrimer increased skin penetration of the therapeutic agent. The invention particularly provides for compositions and methods for transdermal delivery of anticancer and chemo-preventive agents.

### BACKGROUND

The outermost layer of the skin—the stratum corneum (SC) consisting of multiple lipid layers—functions as a protective barrier against exogenous molecules. In particular, the SC layers are excellent barriers against those molecules with molecular weights over 500 g/mol and those with 1-octanol/34 PBS partition coefficients (log P) less than 1 or greater than 3. For this reason, a variety of molecules and materials have been investigated as candidates that enable or facilitate skin permeation of those molecules that are otherwise skin- impermeable. Chemical penetration enhancers (CPEs) have been widely used to increase the skin permeability of many therapeutic molecules and anesthetics (Karade e t al., Proc. Natl. Acad. Sci. USA, 102(3): 4688-4693, 2005). However, the penetration-enhancing effect is frequently accompanied by skin irritation and toxicity. By way of contrast, polymer-based permeation enhancers typically do not cause skin irritation, but their large size often prohibits them from penetrating deep into the skin layers, which limits their efficacy.

Dendrimers are synthetic, spherical macromolecules with tree-like branched structures. Their well-controlled sizes (2-20 nm), ease of functionalization, high water solubility, well-defined chemical structure, and biocompatibility make these nanomaterials attractive for a wide spectrum of promising biomedical applications. Poly(amidoamine) (PAMAM) dendrimers have been shown to be advantageous over linear polymers due to their multivalency, which can be precisely controlled by engineering their surface functional groups. Previously, Hong et al. reported a series of studies on the biological interactions between dendrimers and either lipid bilayers or cell membranes. The studies revealed that positively charged PAMAM dendrimers induce nanoscale hole formation (within noncytotoxic concentrations), whereas neutral or negatively charged PAMAM dendrimers do not. (Hong et al., Chem. Biol. 14(1): 107-115, 2007, Hong et al. Bioconjugate Chem., 17(3): 728-734, 2009, Hong et al., Bioconjugate Chem. 20(8): 1503-1513, 2009). These observations suggested an alternative mechanism of lipid layer permeabilization by positively charged dendrimers, which may be applicable for skin penetration.

A few recent studies have reported that, through emulsion or pretreatment, PAMAM dendrimers enhance, by as much as four-fold, the skin permeability of the nonsteroidal anti-inflammatory drugs (NSAIDs) ketoprofen and diflunisa (Cheng et al. J. Pharm. Sci. 96(3): 595-602, 2007) and the hydrophilic 5-fluorouracil (5FU) (Venuganti et al., Intl. J. Pharma. 361(1-2): 230-238, 2008). It was also reported that permeation of 5FU was enhanced in skin pretreated with generation 4 (G4) or G3.5 PAMAM dendrimers with different n surface functional groups; the order of enhancement in drug permeability coefficient (Kₚ) was G4-NH₂>G4-0H>G3.5-COOH (Venuganti et al. J. Pharma. Sci. 98(1520-6017): 2345-2356, 2008. Meanwhile, the Kₚ of 5FU was inversely proportional to the molecular weight of the dendrimer (Venuganti et al., Intl. J. Pharma. 361(1-2): 230-238, 2008) suggesting that amine-terminated, small PAMAM dendrimers are more effective than other types of dendrimers in enhancing skin permeability of small drug molecules. However, although quite a few reports have shown enhanced skin permeation of small drug molecules mediated by PAMAM dendrimers (Cheng et al. j. Pharm. Sci. 96(3): 595-602, 2007; Venuganti et al., Intl. J. Pharma. 361(1-2): 230-238, 2008; Filipowicz et al., Intl. J. Pharma. 408: 152-156, 2011; Chauhan et al. J. Controlled Release 90(3): 335-343) all of those studies used dendrimer—drug complexes to increase drug solubility and loading. Furthermore, the reported skin permeation was frequently assisted by the addition of CPEs such as mineral oil and isopropyl myristate or by formulating the complex into emulsions using cetyl alcohol and Brij or polysorbate as emulsifiers. More importantly, most of those reports have focused on skin permeation of small molecules, without providing systematic investigations focusing on the interactions of the dendrimers themselves (low generations, in particular) with the skin layers. Further mechanistic studies of how low-generation dendrimers interact with the skin layers are therefore required for better understanding and potential clinical translation of dendrimer-based transdermal drug delivery.

Chemo-preventive drugs for breast cancer have been proven effective in preventing breast cancer development, but are only available for oral administration that causes numerous side effects, which has resulted in low acceptance by the potential patients. The local, transdermal system using dendrimer-drug conjugates of the present invention achieves effective delivery of the drug without systemic exposure, which substantially reduces or even eliminates the adverse effects. For example, endoxifen is a common treatment for ductal carcinoma in situ (DCIS) which is also a possible preventive treatment. However, treatment with endoxifen has many unpleasant side effects when administered orally or intravenously. Thus, there is a need for compositions and methods of transdermal delivery of therapeutic agents that minimize systemic exposure and thereby reduce adverse side effects and increase patient compliance.

### SUMMARY OF INVENTION

The invention provides for dendrimer conjugates that are used for the transdermal delivery of a therapeutic agent. This invention achieves a novel transdermal delivery system of therapeutic agents such as chemopreventive agents, e.g. tamoxifen (TAM) and its metabolites including 4-hydroxytamoxifen (4-OHT) and endoxifen (ENX) as well as other preventive medicine for breast and other types of cancer, comprising a surface-modified PAMAM dendrimer that increase skin penetration of the therapeutic agent.

The studies described in Examples 1-5 investigate the effects of dendrimer size, surface charge, and hydrophobicity as potential key parameters that determine the skin permeation/penetration behavior of dendrimers. The size effect was investigated by comparing G2 and G4 PAMAM dendrimers. The surfaces of G2 PAMAM dendrimers were then modified to be amine-, acetyl-, and carboxyl-terminated to investigate the charge effect as shown in FIG. 1. In addition, G2 PAMAM dendrimers were conjugated with oleic acid (OA) to control the hydrophobicities of the nanomaterials. Using those materials, Franz diffusion cell experiments were carried out. Confocal microscopy observations and partition coefficient analysis were also carried out to assess the dendrimer-skin interactions. This studies provided present a systematic understanding of the interaction between the skin layers and surface-engineered dendrimers, demonstrating the potential of the dendrimers as a transdermal drug delivery vehicle.

Recently, it has been shown that that poly(amidoamine) (PAMAM) dendrimers enhance the translocation efficiency of the drugs across the skin layers. The skin permeation efficiency of the dendrimers was significantly improved by conjugation with the drugs and/or through surface engineering. As described herein in Examples 6 and 7, G2 PAMAM dendrimer conjugated with endoxifen exhibited 30% skin permeation through the porcine skin layers. Further carboxylation of G2 PAMAM dendrimers resulted in up to 5-fold enhancement in skin permeation as compared to the unmodified G2 PAMAM dendrimers. This novel transdermal platform technology provides a novel delivery method not only for breast cancer prevention but also for other disease prevention and treatment, which is safe and yet effective.

The dendrimer conjugates of the invention are small and efficiently penetrate the skin without the need for addition chemical penetration agents (CPE). The small size of dendrimers and the lack of or reduced need for CPE for transdermal delivery of the dendrimer conjugates will reduce skin irritation generally associated with transdermal delivery systems. It was unexpected that small size and surface modifications would increase skin penetration of the therapeutic agent tested (endoxifen) to such a great degree. This enhanced ability to penetrate the skin allows for direct localized administrations to sites in need, e.g. tumor site or the sites where likely will give rise of tumors. Thus, the transdermal delivery systems, e.g. compositions comprising the dendrimer conjugate of the invention, allow for direct localized administration which will decrease systemic exposure to the therapeutic agent and reduce adverse side effects. Furthermore, the dendrimer conjugates can also achieve slow release of the drug conjugated in the skin layers, allowing reduced dosing frequency that will further increase the patience compliance and efficacy.

The invention provides for a dendrimer conjugate comprising a surface-modified G1 to G5 dendrimer associated with a therapeutic agent. The dendrimer conjugates of the invention may comprise any type of . The term “surface modification” refers to modification of the surface groups available on a dendrimer. For example, the surface modified dendrimers may be amine-, acetyl- or carboxyl-terminated. Surface modifications also include conjugating or associating molecules to the surface of the dendrimer, such as attaching a fatty acid such as oleic acid, a label such as a fluorescent dye and/or a therapeutic agent. The molecules may be conjugated or associated with a covalent or a noncovalent bond.

In one embodiment, the invention provides for a dendrimer conjugate comprising a surface-modified G1 to G5 poly(amidoamino) (PAMAM) dendrimer associated with a therapeutic agent wherein the surface of the PAMAM dendrimer is modified to comprise at least 50% carboxylation or at least 50% acetylation and wherein the surface-modified PAMAM dendrimer increases skin penetration of therapeutic agent.

The dendrimer conjugates of the invention may comprises a PAMAM dendrimer ranging in size from G1 to G5 PAMAM dendrimers, G1 to G2 dendrimers, G2 to G3 dendrimers, G2 to G4 dendrimers, G2 to G5 dendrimers, G1.5 to G3 dendrimers, G2.0 to G2.9 dendrimers, G2.0 to G3.9 dendrimers, or G1.5 to G3.5 dendrimers, or G2.5 to G3.5 dendrimers, or G3 to G5 dendrimers, or G3 to G4 dendrimers, or G4 to G5 dendrimers. For example, the dendrimer conjugate may comprise a dendrimer selected from the group consisting of G1 PAMAM dendrimer, G2 PAMAM dendrimer, G3 PAMAM dendrimer, G4 PAMAM dendrimer and G5 PAM AM dendrimer.

In any of the dendrimer conjugates of the invention, the dendrimer may comprise, be administered with or is mixed with a penetration enhancer (CPE). The CPE may be selected from the group consisting of fatty acids, fatty alcohols, fatty acid esters, fatty alcohol ethers, biologics, enzymes, amines, amides, complexing agents, ionic compounds, dimetyl sulfoxide, N-methyl pyrrolidone, polar solvents, salicyclic acid, benzyl nicotinate, azones, polyhydric alcohols, oils, fatty ethers, urea, and surfactants or combinations thereof. In particular, the invention provides for dendrimer conjugates wherein the PAMAM dendrimer comprises, is mixed with or is administered with a CPE that is a fatty acid, such as oleic acid.

In any of the dendrimer conjugates of the invention, the dendrimer, such as a PAMAM dendrimer, and the therapeutic agent may be in a physical mixture. The term “physical mixture” refers to the physical combination of two or more substances. The physical mixture is the result of mechanical blending or mixing without chemical binding or other chemical changes so that the each substance retains its own chemical properties. A physical mixture may be in the form of a solution, suspension, colloid or alloy.

In addition, in any of the dendrimer conjugates of the invention, the therapeutic agent is attached or associated to the dendrimer, such as a PAMAM dendrimer. For example, the therapeutic agent may be covalently associated to the PAMAM dendrimer or the therapeutic agent may be associated to the PAMAM dendrimer by a noncovalent bond. In particular, the invention provides for dendrimer conjugates wherein the therapeutic agent is associated with the PAMAM dendrimer by an amide bond.

The dendrimer conjugates of the invention comprise a dendrimer, such as a PAMAM dendrimer, associated with a therapeutic agent, wherein the therapeutic agent is selected from the group consisting of anticancer agents, chemopreventive agents, anesthetics, anorexics, anti-allergics, antiarthritics, antiasthmatic agents, antibiotics, anticholinergics, anticonvulsants, antidepressants, antihemophilics, antidiabetic agents, antidiarrheals, antifungals, antigens, antihistamines, antihypertensives, anti-inflammatories, antimigraine preparations, antinauseants, antineoplastics, antiparkinsonism drugs, antiprotozoans, antipruritics, antipsychotics, antipyretics, antispasmodics, antivirals, calcium channel blockers, cardiovascular preparations, central nervous system stimulants, contraceptives, cough and cold preparations including decongestants, diuretics, enzyme inhibitors, enzymes, genetic material including DNA and RNA, growth factors, growth hormones, hormone inhibitors, hypnotics, immunoactive agents, immunosuppressive agents, microbicides, muscle relaxants, parasympatholytics, peptides, peripheral and cerebral vasodilators, proteins, psychostimulants, receptor agonists, sedatives, spermicides and other contraceptives, steroids, sympathomimetics, tranquilizers, vaccines, vasodilating agents including general coronary, viral vectors and small organic molecules or a combination thereof.

In particular, the invention provides for a dendrimer conjugate wherein the dendrimer, such as a PAMAM dendrimer, is associated with a chemopreventive agent, such as tamoxifen, endoxifen, 4-hydroxytamoxifen, raloxifene, 4-hydroxyphenylretinamide (fenertinide), finasteride and other drugs that reduce the amount of dihydrotestosterone , asparin, non-seroidal anti-inflammatory drugs (NSAIDS) such as selective COX-2 inhibitors such as celecoxib, selective estrogen receptor modulators (SERMS) such as LY35381-HCl, selective binder to retinoid X receptors (rexinoids) such as LG 100268 and targretin (LGD 1069), peroxisome proliferator-activated receptor γ (PPAR-γ) ligands such as GW 7845. Chemopreventive agents are therapeutic agents that lower the risk of cancer or slow its development by preventing tumor growth or reducing the risk of tumor growth.

In another aspect, the invention provides for compositions comprising any of the dendrimer conjugates of the invention and a carrier. In particular, the invention provides for compositions comprising any of the dendrimer conjugates of the invention and a carrier formulated for transdermal delivery. In any of the compositions of the invention, the carrier may be a liquid, gel, solvent, liquid diluents, solubilizer, hydrogel, paraffin, wax, oil, silicone, ester, oily cream, aqueous cream, water soluble base, glycerol, glycol, lotion , polymer, powder or microemulsion.

Any of the compositions of the inventions may further comprise a chemical penetration enhancer (CPE). Within the composition, the CPE may be associated with the dendrimer, such as a PAMAM dendrimer, by a covalent or noncovalent bond. In addition, the CPE may be in a physical mixture with the dendrimer conjugate within the composition. The CPE may be selected from the group consisting of fatty acids, fatty alcohols, fatty acid esters, fatty alcohol ethers, biologics, enzymes, amines, amides, complexing agents, ionic compounds, dimetyl sulfoxide, N-methyl pyrrolidone, polar solvents, salicyclic acid, benzyl nicotinate, azones, polyhydric alcohols, oils, fatty ethers, urea, and surfactants or combinations thereof. In particular, the invention provides for dendrimer conjugates wherein a PAMAM dendrimer comprises, is mixed with or is administered with a CPE that is a fatty acid, such as oleic acid.

In addition, any of the compositions of the invention may be attached to or within a device for transdermal delivery. The devices include patches, gauze, pressure sensitive adhesives, adhesive bandages, microchips or microneedles.

In another aspect of the invention, the invention provides for methods of transdermal delivery of a therapeutic agent to a subject comprising contacting a surface modified G1 to G5 poly(amidoamino) (PAMAM) dendrimer associated with the therapeutic agent with the skin of the subject, wherein the surface of the PAMAM dendrimer is modified to comprise at least 50% carboxylation or at least 50% acetylation, and wherein the surface modified PAMAM dendrimer increases penetration of the therapeutic agent into the skin of the subject.

The invention also provides for method for transdermal delivery wherein the contacting of the dendrimer with the skin of the subject occurs at a site of need in the subject. The term “site of need” is the location where the disease state, injury or disorder is prominent in the subject or the site on the skin that is in closest proximity to the where the disease state or disorder is prominent in the subject, or the site on the skin that provides an accessible route to the location where the disease state, injury or disorder is prominent, or the site where a cause of the disease state, injury or order is located. For example, the site of need may be in a location where tumors tend to develop. In another example, for a subject at risk of developing breast cancer or a subject that is suffering from breast cancer, the site of need is on the breast.

The invention also provides for methods of transdermal delivery of a therapeutic agent to a subject wherein the transdermal delivery of the therapeutic agent decreases systemic exposure of the therapeutic agent in the subject.

In addition, the invention provides for methods of transdermal delivery of a therapeutic agent to the subject wherein transdermal delivery of the therapeutic agent decreases adverse effects of the therapeutic agent on the subject.

Further, the invention provides for methods of transdermal delivery of a therapeutic agent to a subject wherein the transdermal delivery of the therapeutic agent increases compliance by the subject.

In one embodiment, the invention provides for methods of decreasing systemic exposure of a therapeutic agent in a subject comprising administering a surface modified G1 to G5 poly(amidoamino)(PAMAM) dendrimer associated with a therapeutically effective dose of a therapeutic agent on the skin of the subject at a site of need, wherein the surface of the PAMAM dendrimer is modified to comprise at least 50% carboxylation or at least 50% acetylation and wherein the surface modified PAMAM dendrimer increases skin penetration of therapeutic agent and wherein the therapeutic agent is administered at a dose that is less than a therapeutically effective dose of the therapeutic agent administered orally or intravenously.

In another embodiment, the invention provides for methods of decreasing adverse effects of a therapeutic agent in a subject comprising administering a surface modified G1 to G5 poly(amidoamino) (PAMAM) dendrimer associated with a therapeutically effective dose of a therapeutic agent on the skin of the subject at a site of need, wherein the PAMAM dendrimer is modified to comprise at least 50% carboxylation or at least 50% acetylation and wherein the surface modified PAMAM dendrimer increases skin penetration of therapeutic agent and wherein the therapeutic agent is administered at a dose that is less than the therapeutically effective dose of the therapeutic agent administered orally or intravenously.

In a further embodiment, the invention provides for methods of reducing the risk of or preventing tumor growth in a subject in need comprising administering a surface modified G1 to G5 poly(amidoamino) (PAMAM) dendrimer associated with a therapeutically effective dose of a chemopreventive agent on the skin of the subject at a site of need, wherein the PAMAM dendrimer is modified to comprise at least 50% carboxylation or at least 50% acetylation and wherein the surface modified PAMAM dendrimer increases skin penetration of therapeutic agent, such as methods wherein the chemopreventive agent is tamoxifen, endoxifen or 4-hydroxytamoxifen.

### BRIEF DESCRIPTION OF DRAWINGS

FIG. 1 depicts the chemical structure of G2 PAMAM dendrimer. The representative structure of the surface modified dendrimers: (A) amine-, (B) carboxyl-, and (C) acetyl-terminated dendrimers.

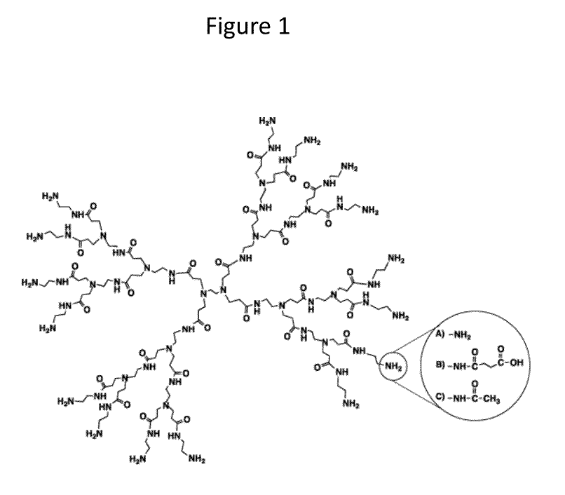

FIG. 2A-2B depicts reaction schemes of the surface modifications of G2 PAMAM dendrimers: (A) Conjugation of G2-RITC-NH2 (same conditions for G4-RITC-NH₂ conjugation) and surface charge modification by acetylation and carboxylation of G2-RITC-NH₂. (B) Conjugation of OA to G2-RITC [symbol] NH₂.

FIG. 3A-3B depicts skin permeation efficiencies of various PAMAM dendrimers. (A) shows a Fold increase in epidermal accumulations of G2 and G4 PAMAM dendrimers over 24 hours (error bars: standard deviation (SD), n=3). (B) Fold enhancement in % permeation of the surface-modified G2 PAMAM dendrimers after 24 h (error bars: SD, n=3-6). *p<0.05.

FIG. 4A-4B depicts the relationships between hydrophobicity and skin retention of the surface-modified G2 dendrimers: (A) Partition coefficients of various G2 PAMAM dendrimers measured using the shake-flask method. The experimental groups include G2-RITC-COOH, G2-RITC-Ac, G2-RITC-NH₂, and two types of OA-conjugated dendrimers: G2-RITC-NH₂-OA_{2.3} and G2-RITC-NH₂-OA_{2.7}. The observed higher skin deposition of the G2-RITC-NH₂-OA conjugates is likely a result of their higher partition coefficients than other types of dendrimers. (B) Skin deposition and retention of various G2 PAMAM dendrimers and their conjugates after 24 h of the Franz cell experiment (error bars: standard error from the donor solution, n=3)

FIG. 5 depicts the reaction scheme of the attachment of endoxifen to G2 PAMAM dendrimers via amide bond.

FIG. 6 demonstrates that the G2-RITC-EDX conjugate greatly enhanced permeation through porcine skin.

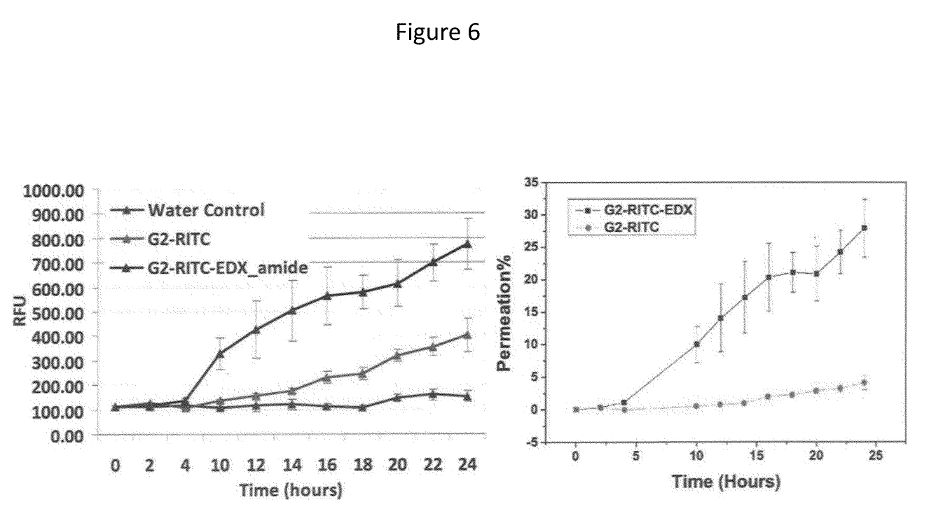

FIG. 7 depicts % permeation of the surface modified PAMAM dendrimers.

### DETAILED DESCRIPTION

Although several reports have studied PAMAM dendrimers as a potential skin penetration enhancer (Cheng et al. j. Pharm. Sci. 96(3): 595-602, 2007; Venuganti et al., Intl. J. Pharma. 361(1-2): 230-238, 2008; Filipowicz et al., Intl. J. Pharma. 408: 152-156, 2011; Chauhan et al. J. Controlled Release 90(3): 335-343; Borowska et al., Intl. J. Pharma 398: 185-189, 2010), the skin permeation and retention behaviors of PAMAM dendrimers themselves are largely unknown. Therefore, in the experiment described herein, are designed to reveal the role of size, surface charge, and hydrophobicity of dendrimers in the skin permeation/deposition of the materials in a systematic manner. The studies described in examples 1-6 investigates three hypotheses (i) smaller dendrimers penetrate the skin better than larger ones; (ii) surface modification of G2 PAMAM dendrimers enhances or alters skin permeability; and (iii) the partition coefficient (hydrophobicity) determines the permeation efficiency of the dendrimer conjugates.

The experiments described herein demonstrate that physicochemical properties of PAMAM dendrimers directly affect the skin interactions of the macromolecules. To summarize, amine-terminated dendrimers are beneficial for the localized transdermal delivery given their enhanced skin deposition and retention. Acetyl- or carboxyl-terminated dendrimers are more effective for systemic delivery through topical administration, given their enhanced permeation. Furthermore, smaller dendrimers exhibit enhanced skin permeation and strong dendrimer-skin interactions, particularly when their hydrophobicity is optimized through conjugation with hydrophobic molecules such as drug molecules. In addition to the potential application of the surface-modified dendrimers for transdermal delivery, the modularity in surface engineering enables them to be applied for controlled intestinal absorption after oral administration. The paracellular and transcellular permeation pathways, observed using Caco-2 cells (Lin et al. Nanoscale 2(5): 755-762, 2010) well-correlate to the results presented herein, indicating the potential of the surface-engineered dendrimers to overcome the challenge of low permeability through the intestinal barriers.

### Dendrimers

The term “dendrimer” refers to repeatedly branched nano-sized macromolecules characterized by a symmetrical, well-defined three-dimensional shape. Dendrimers grow three-dimensionally by the addition of shells of branched molecules to a central core. The cores are spacious and various chemical units can be attached to points on the exterior of the central core. Dendrimeric polymers have been described extensively (Tomalia. (1994). Advanced Materials 6:529-539; Donald A. Tomalia, Adel M. Naylor, William A. Goddard III (1990). Angew, Chem. Int. Ed. Engl., 29:138-175; incorporated herein by reference in their entireties). Dendrimers are synthesized as defined spherical structures typically ranging from 1 to 20 nanometers in diameter. Accordingly, in some aspects, the dendrimers of the dendrimer conjugates provided herein are about 1, about 2, about 3, about 4, about 5, about 6, about 7, about 8, about 9, about 10, about 11, about 12, about 13, about 14, about 15, about 16, about 17, about 18, about 19, or about 20 nm in diameter.

Dendrimers are synthetic, monodispersed branched macromolecules that form a tree-like structure whose synthesis represents a relatively new field of polymer chemistry. PAMAM dendrimers, first synthesized using the divergent synthetic route, have shown promise for biomedical applications due to their: i) well-controlled size (3 to 10 nm); ii) ease of functionalization (conjugation with biofunctional molecules such as imaging agents and drugs); iii) high water solubility; iv) well-defined chemical structure; and v) biocompatibility. PAMAM dendrimers offer distinct advantages over linear polymers due to the multivalency and precision with which the number of surface functional groups can be modified, by controlling the number of branching units (Hong et al. Chem. Biol. 14: 107-115, 2007).

A few recent studies have reported that PAMAM dendrimers enhance the skin permeability of nonsteroidal anti-inflammatory drugs (NSAIDs) such as Ketoprofen and Diflunisal, as well as of model hydrophilic drugs such as 5-fluorouracil (5-FU) (Cheng et al., 96: 595-602, 2007, Venuganti et al. Intl. J. Pharma. 361: 203-238, 2008) . Up to 4-fold enhancement in permeability coefficient of 5-FU was observed after 24 hours of dendrimer pre-treatment, followed by application of 5-FU in isopropyl myristate (IpM) for 48 hrs (, Venuganti et al. Intl. J. Pharma. 361: 203-238, 2008; Venuganti et al., J. Pharma. Sci. 2008) . Similarly, the accumulative permeated amounts of Ketoprofen and Diflunisal through excised rat skin layers were increased by 3.4 and 3.2 times, respectively, by complexing with PAMAM dendrimers (after 24 hours of co-treatment), as compared to the free drugs (Cheng et al., 96: 595-602, 2007). There are several dendrimer properties that have been considered to contribute to the observed skin permeation: i) high water solubility of PAMAM dendrimers increases bioavailability of drugs and ii) the cationic nature strongly interact with the skin surface that are negatively charged, which alters the skin permeability and facilitates complexed drugs to reach the skin surface. However, the mechanisms of the dendrimer-mediated skin permeabilization are not well understood, and may not require penetration of the dendrimer molecules through the skin. The present invention provides a surface modified small dendrimer (e.g. G1-G5) that efficiently penetrates the skin.

Dendrimers are identified by a generation number (Gn) and each complete synthesis reaction results in a new dendrimer generation. Molecular weight and the number of terminal groups increase exponentially as a function of generation number (the number of layers) of the dendrimer. Different types of dendrimers can be synthesized based on the core structure that initiates the polymerization process. Dendrimers of any generation are used for the invention. For example, the present invention provides for G1, G2, G3, G4 and G5 dendrimers

The dendrimer core structures in some aspects dictate several characteristics of the molecule such as the overall shape, density and surface functionality (Tomalia et al. (1990). Angew. Chem. Int. Ed. Engl., 29:138). Spherical dendrimers have ammonia as a trivalent initiator core or ethylenediamine (EDA) as a tetravalent initiator core. Recently described rod-shaped dendrimers (Yin et al (1998). J. Am. Chem. Soc., 120:2678) use polyethyleneimine linear cores of varying lengths; with longer cores leading to increased rod length. Dendritic macromolecules are available commercially in kilogram quantities and are produced under current good manufacturing processes (GMP) for biotechnology applications.

As used herein, the term “dendrimer” also refers to unsymmetrical or asymmetrical dendrimers having more than one radius due to asymmetry of the dendrimer. In some aspects, the asymmetrical dendrimer has two different radii. Such dendrimers and the synthesis thereof are further described in Lee et al., Bioconjugate Chem. 18: 579-584 (2007).

Dendrimers may be characterized by a number of techniques including, but not limited to, electrospray-ionization mass spectroscopy, matrix-assisted laser desorption/ionization—time of flight spectroscopy, ¹³C nuclear magnetic resonance spectroscopy, high pressure liquid chromatography, size exclusion chromatography with multi-angle laser light scattering, capillary electrophoresis and gel electrophoresis. These tests assure the uniformity of the polymer population and are important for monitoring quality control of dendrimer manufacture for GMP applications and in vivo usage. Extensive studies have been completed with neutralized dendrimers and show no evidence of toxicity when administered intravenously in vivo.

The invention contemplates the use of any type of dendrimer including but not limited to poly(amidoamine) (PAMAM) dendrimers such as dense star polymers and Starburst polymers, poly(amidoamine-organo silicon) (PAMAMOS) dendrimers, (Poly (Propylene Imine)) (PPI) dendrimers, tecto dendrimers, multilingual dendrimers, chiral dendrimers, hybrid dendrimers/linear polymers, amphiphilic dendrimers, micellar dendrimers and Fréchet-type dendrimers.

In one embodiment, the dendrimer conjugate comprises a PAMAM dendrimer. PAMAM dendrimers are a family of water-soluble polymers characterized by a unique tree-like branching architecture and a compact spherical shape in solution. Several classes of PAMAM dendrimers have been synthesized using different cores such as ethylene diamine (EDA) and 1,4-diamino butane (DAB) with different surface groups (e.g. amine, hydroxyl, or carboxyl). PAMAM dendrimers are identified by a generation number (Gn) in the range 0-10 where an increase in Gn denotes a controlled incremental increase in size, molecular weight, and number of surface groups. PAMAM dendrimers are efficient drug carriers due to the high degree of branching and the large number of surface groups, which can be utilized to immobilize drugs, imaging agents, or targeting ligands to achieve a high density of therapeutic molecules in a compact system.

PAMAMOS dendrimers are composed of radially layered poly(amidoamine-organosilicon) units. These dendrimers are inverted unimolecular micelles that consist of hydrophilic, nucleophilic PAMAM interiors and hydrophobic organosilicon (OS) exteriors. These dendrimers may serve as precursors for the preparation of honeycomb-like networks with nanoscopic PAMAM and OS domains.

PPI dendrimers are generally poly-alkyl amines having primary amines as terminal groups. The PPI dendrimer interior consists of numerous tertiary tris-propylene amines. PPI dendrimers are also known as POPAM (Poly (Propylene Amine) with DAB cores.

Tecto dendrimers are composed of a core dendrimer, surrounded by dendrimers of differing type in order to impart specific regional functionality in the smart therapeutic nanodevice. Different compounds perform varied functions ranging from diseased cell recognition, diagnosis of disease state drug delivery, reporting location to reporting outcomes of therapy. Multilingual dendrimers are dendrimers in which the surface contains multiple copies of a particular functional group. Chiral dendrimers are based upon the construction of constitutionally different but chemically similar branches to chiral core. Hybrid dendrimers/linear polymers are hybrids (block or graft polymers) of dendritic and linear polymers. Amphiphilic dendrimers are dendrimers that have two segregated sites of chain end, one half is electron donating and the other half is electron withdrawing. Micellar dendrimers are unimolecular micelles of water soluble hyper branched polyphenylenes.

Fréchet-Type dendrimers are based on a poly-benzyl ether hyper-branched skeleton. These dendrimers usually have carboxylic acid groups as surface groups, serving as a good anchoring point for further surface fictionalization, and as polar surface groups to increase the solubility of this hydrophobic dendrimer type in polar solvents or aqueous media.

### Transdermal Delivery and Formulations

The “transdermal delivery” is intended both transdermal (or “percutaneous” or “dermal”) and transmucosal administration, i.e., delivery by passage or penetration of a drug through the skin or mucosal tissue and into the bloodstream.

The term, “flux” (also called “permeation rate”) is defined as the absorption of the drug through the skin or mucosa, and is described by Fick's first law of diffusion: J=-D(dCm/dx), where J is the flux in g/cm²/sec, D is the diffusion coefficient of the drug through the skin or mucosa in cm2/sec and dCm/dx is the concentration gradient of the drug across the skin or mucosa.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as an ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope of this invention.

An example transdermal formulation is: stearyl alcohol (250 g) and a white petrolatum (250 g) are melted at about 75°. and then a mixture of a dendrimer conjugate of the of the invention, methylparaben (0.25 g), propylparaben (0.15 g), sodium lauryl sulfate (10 g), and propylene glycol (120 g) dissolved in water (about 370 g) is added and the resulting mixture is stirred until it congeals.

In one embodiment, the formulations herein can be in the form of aqueous gel, anhydrous gel, a water-in-oil emulsion, oil-in-water emulsion or a suspension. Examples of gel forming procedure for DHEA can be found in U.S. Pat. Nos. 5,709,878, and 4,978,532 the entire content of which are incorporated by reference herein. Gels are semisolid systems of either containing suspended small inorganic particles (two phase gels) or organic macromolecules interpenetrated by a liquid (single phase gels). Emollients such as petrolatum, paraffin wax, beeswax, cetyl palmitate, and lanolin can be included in the formulations herein. When formulated for presentation as a gel, the composition of the invention can include a gelling agent such as a finely divided solid and/or a thickener in concentrations that produce a loose molecular network inhibiting the free movement of liquid ingredients. Thus a typical gel composition of the invention includes a concentration of dendrimer conjugate in the range of about 0.1 to about 20 grams per 100 grams of composition, preferably about 0.25 to about 5 grams per 100 grams; a concentration of phospholipid in the range of about 2 to about 50 grams per 100 grams of composition, preferably about 3 to about 25 grams per 100 milliliters; a concentration of finely divided solid in the range of about 0 to about 15 grams per 100 grams of composition, and a concentration of thickener in the range of about 0 to about 15 grams per 100 grams of composition.

Gellants may also be included in the formulations. These agents are typically non-ionic or cationic polymers such as hydroxyethyl cellulose, methylcellulose, guar gum, xanthan gum, hydroxypropylcellulose and cationic cellulosics. A particular example is Sepigel. In one embodiment, a gel comprising a dendrimer conjugate, can be made by mixing a lower alkyl alcohol, a polysorbate, water and a dendrimer conjugate and, optionally, adding and mixing a thickening agent followed by incubating the ingredients until gel formation. Various temperatures may be used for incubation to effect gel formation. A preferred temperature range is about 3° C. to about 90° C.; a more preferred range is about 10° C. to about 50° C.; and more preferred range is about 10° C. to about 40° C. Incubation times vary depending on the temperature, and the ratio of ingredients. The ratios of ingredients may also vary depending on the particular therapeutic agent associated to the dendrimer within the dendrimer conjugate and the particular lower alcohol use. The composition may comprise alcohol in the range of from about 20 to about 95% (v/v); preferably from about 30 to about 90%; even more preferably about 50 to about 90%. The water content may from about 0 to about 60%; preferably about 2 to about 40%; more preferably about 5 to about 30%; even more preferably about 15 to about 30%. The surfactant may be present in the range of about 0 to 10%; more preferably about 0.01% to about 5%; even more preferably about 0.01% to about 3.5%.

Examples of thickening agents that can be added to the gel or solution formulations described herein include: cellulosic thickening agents, for example, cellulose, hydroxyethyl-cellulose, carboxymethylcellulose, and hydroxypropylmethyl-cellulose; and acrylic thickening agents. Examples of preferred acrylic thickeners are carbomers, for example, non-linear polymers of acrylic acid cross-linked with a polyalkenyl polyether. Examples of preferred carbomers which may be used in the present invention include carboxypolymethylene, carboxyvinyl polymer, and alkyl acrylates, for example, acrylic acid/alkyl methacrylate copolymer. All of the above are available from Noveon, with carboxypolymethylene sold as Carbopol 980 carboxyvinyl polymer sold as Carbopol 940, and acrylic acid/alkyl methacrylate copolymer sold as Pemulen TR-1.

In a preferred embodiment, the formulations of the invention can be applied by misting or spraying the formulation on the skin either via a metered dose device or from a unit dose container. In this method, the formulation can be distributed evenly over a larger area thereby providing a quick means for absorption. Alternatively the formulation can be applied via an applicator, such as a roll-on applicator, a metered pump dispenser or sponge.

A topical oil-in-water emulsion composition can be prepared by making a solution of fluasterone (or related compound) as described above and adding an immiscible phase (e.g., a biocompatible oil phase) and an optional emulsifying agent. An irritation mitigating agent can also be included, such as C₁₂₋₁₅ alkyl benzoate, octyl methoxycinnamate, octyl dimethyl PABA, octocrylene, menthyl anthranilate, and homomenthyl salicylate.

In certain preferred embodiments a foam comprising compounds of instant application can be prepared. An example of a foam forming procedure can be found in U.S. Pat. No. 7,141,237. For instance, an active agent in a solution as described herein and a quick-breaking foaming agent comprising a mixture of cetyl alcohol and stearyl alcohol, which are dissolved in the ethanol solution can be used. Preferably, this composition is packaged in a polyamide-imide-lined aluminum can and pressurized with a propane/butane mixture as the propellant. Under the packaged pressure, the hydrocarbon propellant liquefies and becomes miscible with the water/ethanol solution.

The formulation herein may contain an emulsifier and/or surfactant. A wide variety of such agents can be employed. In one embodiment, the compositions of the present invention comprise from about 0.05% to about 95%, preferably from about 10% to about 80%, and more preferably from about 3.5% to about 60% of at least one surfactant. The surfactant, at a minimum, must be hydrophilic enough to disperse in ethanol or other solvent system. The surfactants useful herein can include any of a wide variety of cationic, anionic, zwitterionic, and amphoteric surfactants disclosed in prior patents and other references. The exact surfactant chosen will depend upon the pH of the composition and the other components present.

In one embodiment, the composition comprises a hydrophilic emulsifier or surfactant. The compositions of the present invention preferably comprise from about 0.05% to about 5%, more preferably from about 0.05% to about 3.5% of at least one hydrophilic surfactant. Without intending to be limited by theory, it is believed that the hydrophilic surfactant assists in dispersing hydrophobic materials.

Preferred hydrophilic surfactants are selected from nonionic surfactants. Among the nonionic surfactants that are useful herein are those that can be broadly defined as condensation products of long chain alcohols, e.g. C8-30 alcohols, with sugar or starch polymers, i.e., glycosides. These compounds can be represented by the formula (S)ₙ—O—R wherein S is a sugar moiety such as glucose, fructose, mannose, and galactose; n is an integer of from about 1 to about 1000, and R is a C8-30 alkyl group. Examples of long chain alcohols from which the alkyl group can be derived include decyl alcohol, cetyl alcohol, stearyl alcohol, lauryl alcohol, myristyl alcohol, oleyl alcohol, and the like. Preferred examples of these surfactants include those wherein S is a glucose moiety, R is a C8-20 alkyl group, and n is an integer of from about 1 to about 9. Commercially available examples of these surfactants include decyl polyglucoside and lauryl polyglucoside.

Other useful nonionic surfactants include the condensation products of alkylene oxides with fatty acids (i.e. alkylene oxide esters of fatty acids); the condensation products of alkylene oxides with 2 moles of fatty acids (i.e. alkylene oxide diesters of fatty acids); the condensation products of alkylene oxides with fatty alcohols (i.e. alkylene oxide ethers of fatty alcohols); and the condensation products of alkylene oxides with both fatty acids and fatty alcohols. Nonlimiting examples of these alkylene oxide derived nonionic surfactants include ceteth-6, ceteth-10, ceteth-12, ceteareth-6, ceteareth-10, ceteareth-12, steareth-6, steareth-10, steareth-12, PEG-6 stearate, PEG-10 stearate, PEG-100 stearate, PEG-12 stearate, PEG-20 glyceryl stearate, PEG-80 glyceryl tallowate, PEG-10 glyceryl stearate, PEG-30 glyceryl cocoate, PEG-80 glyceryl cocoate, PEG-200 glyceryl tallowate, PEG-8 dilaurate, PEG-10 distearate, and mixtures thereof.

Other nonionic surfactants suitable for use herein include sugar esters and polyesters, alkoxylated sugar esters and polyesters, C1-C30 fatty acid esters of C1-C30 fatty alcohols, alkoxylated derivatives of C1-C30 fatty acid esters of C1-C30 fatty alcohols, alkoxylated ethers of C1-C30 fatty alcohols, polyglyceryl esters of C1-C30 fatty acids, C1-C30 esters of polyols, C1-C30 ethers of polyols, alkyl phosphates, polyoxyalkylene fatty ether phosphates, fatty acid amides, acyl lactylates, and mixtures thereof. Nonlimiting examples of these non-silicon-containing emulsifiers include: polyethylene glycol 20 sorbitan monolaurate (Polysorbate 20), polyethylene glycol 5 soya sterol, Steareth-20, Ceteareth-20, PPG-2 methyl glucose ether distearate, Ceteth-10, Polysorbate 80, cetyl phosphate, potassium cetyl phosphate, diethanolamine cetyl phosphate, Polysorbate 60, glyceryl stearate, polyoxyethylene 20 sorbitan trioleate (Polysorbate 85), sorbitan monolaurate, polyoxyethylene 4 lauryl ether sodium stearate, polyglyceryl-4 isostearate, hexyl laurate, PPG-2 methyl glucose ether distearate, PEG-100 stearate, and mixtures thereof. Commercially available surfactants include polysorbate 80 (Tween 80), polysorbate 20 (Tween 20), polysorbate 40 (Tween 40) and polysorbate (60). The preferred surfactants include polysorbates and more preferred surfactant is Tween 80.

The dendrimer conjugates and compositions of this invention can also be administered by a transdermal device. Accordingly, transdermal administration can be accomplished using a patch either of the reservoir or porous membrane type, or of a solid matrix variety.

A device, or individual dosage unit, of the present invention can be produced in any manner known to those of skill in the art. After the dermal composition is formed, it may be brought into contact with the backing layer in any manner known to those of skill in the art. Such techniques include calender coating, hot melt coating, solution coating, etc. Of course, backing materials are well known in the art and can comprise plastic films of polyethylene, vinyl acetate resins, ethylene/vinyl acetate copolymers, polyvinyl chloride, polyurethane, and the like, metal foils, non-woven fabric, cloth and commercially available laminates. The backing material generally has a thickness in the range of 2 to 1000 micrometers and the dermal composition is generally disposed on backing material in a thickness ranging from about 12 to 250 micrometers thick.

Suitable release liners are also well known in the art and include the commercially available products of Dow Corning Corporation designated Bio-Release7. liner and Syl-off7 7610 liner. For preferred embodiments in which a polysiloxane is part of the multiple polymeric adhesive carrier, the release liner must be compatible with the silicone adhesive. An example of a suitable commercially available liner is 3M's 1022 Scotch Pak.7 The configuration of the transdermal delivery system of the present invention can be in any shape or size as is necessary or desirable. Illustratively, a single dosage unit may have a surface area in the range of 1 to 200 cm². Preferred sizes are from 5 to 60 cm².

In a preferred method aspect of the invention where the carrier is a flexible, finite polymer, one or more polymers are blended, optionally with PVP to result in a pressure-sensitive adhesive composition, or transdermal drug delivery system adhesive system (with incorporated parent drug:prodrug), which controls delivery of an incorporated parent drug:prodrug and through the epidermis. In a preferred embodiment of the invention, a transdermal drug delivery system is prepared by mixing a soluble PVP, polyacrylate, polysiloxane, parent drug/prodrug, optional enhancer(s), co-solvent(s), and tackifying agents, if needed, in an appropriate volatile solvent(s), then casting the mixture and removing the solvent(s) by evaporation to form a film. Suitable volatile solvents include, but are not limited to, alcohols such as isopropanol and ethanol; aromatics such as xylenes and toluene; aliphatics such as hexane, cyclohexane, and heptane; and alkanoic acid esters such as ethyl acetate and butyl acetate.

Included are delivery systems for transdermal administration by: passive patches, heated passive patches, passive patches applied onto RF treated skin, and spray-on-skin systems where the total amount applied is fixed and delivery is improved by co-formulated permeation enhancers.

In one embodiment, the composition of this invention is administered to the recipient by means of a transdermal delivery system or patch. Transdermal delivery is accomplished by exposing a source of the substance to be administered to the recipient's skin for an extended period of time. Typically, the formulation is incorporated in or absorbed on a matrix or container from which it is released onto the recipient's skin. The rate of release can be controlled by a membrane placed between the container and the skin, by diffusion directly from the container, or by the skin itself serving as a rate-controlling barrier. Many suitable transdermal delivery systems and containers therefore are known, ranging in complexity from a simple gauze pad impregnated with the substance to be administered and secured to the skin with an adhesive bandage to multilayer and multi-component structures. Some of the systems are characterized by the use with the substance to be administered of a shaped article sufficiently flexible to snugly fit to the skin of the recipient and thus serve both as container from which the substance is delivered to the recipient's skin and as barrier to prevent loss or leakage of the substance away from the area of the skin to which the substance is to be delivered. A transdermal delivery system or patch may also contain an added substance that assists the penetration of the active ingredient through the skin, usually termed a skin enhancer or penetration enhancer. Transdermal delivery systems may contain an ethoxylated oil such as ethoxylated castor oil, ethoxylated jojoba oil, ethoxylated corn oil, and ethoxylated emu oil. An alcohol mixed with the ethoxylated oil may form a penetration enhancer.

One advantage of transdermal systems is an ability to provide a sustained release of medication over time, which may serve to provide a longer duration of action. However, a significant limitation and disadvantage of passive transdermal administration is a slow onset of sufficient action to provide relief. It is not uncommon for a passive transdermal patch to take several hours (3 or more) before a therapeutic dosage is achieved. With passive transdermal delivery, the skin can act as a depot, and release to the bloodstream will not occur until that skin depot area is saturated. This slow onset of action acts as a clinical limitation in two respects: 1) it cannot replace an existing oral or injectable form because it is a necessity to apply a patch several hours prior to a chemotherapy or operative procedure, and 2) a slow acting transdermal patch cannot reasonably serve as a rescue medication form, where a patient will prefer, for obvious reasons, a faster acting treatment. This second limitation is significant, in that it has been shown that, in many cases of highly emetogenic therapies, such as high dose chemotherapy, a significant percentage of patients will not be adequately served by a first, primary dosage form alone.

A more rapid onset of action can be achieved transdermally by using a system that includes iontophoresis. Granisetron in its hydrochloride salt form, is positively charged and can be delivered rapidly from a positively charged anode pad. Recent reports, for example, Scientific Abstract 1: Evaluation of iontophoretic permeation kinetics of granisetron through skin by subcutaneous microdialysis, presented at the 2003 AAPS meeting October, 2003; Scientific Abstract 2: IVIVC of Iontophoretic Delivery of granisetron by subcutaneous microdialysis, presented at the 2004 AAPS meeting October, 2004, have demonstrated that with iontophoresis, a therapeutic dosage can be achieved (in a hairless rat animal model) within approximately two-hours.

The two-hour system described in the reports, however, is not likely to provide additional benefit for emesis which may occur for up to several days after an exposure to an emetogenic procedure. Additionally, even the two-hour timeframe for achievement of a therapeutic dosage level is also an unacceptably long period of time necessary for clinician and patient to be waiting prior to an emetogenic treatment such as chemotherapy. Finally, the known iontophoresis patches do not provide a means to administer a second or rescue dosage for emesis management in the event the primary dosing from the patch is inadequate.

Therefore, a need exists for a simple-to-operate, inexpensive transdermal dosage form which can not only provide benefit afforded by a transdermal release of agents such as granisetron, but can also provide an initial or primary dose and one or more follow-on self-administered rescue doses treatment very rapidly.

Other mechanical methods of enhancement and delivery of pharmaceutical drugs transdermally include: physical therapy (e.g., massage), electroporation, transdermal patches, implantable release devices/microchips, microneedle injection arrays, needleless injection devices, chemical or physical skin peels (microdermabrasion), magnetophoresis, and laser-radiation photomechanical wave devices. These other methods may form a basis for further disclosure at some other time.

The invention also provides for transdermal delivery systems wherein the dendrimer conjugate is administered in combination with liposomes, niosomes or elastic vesicles such as transferosomes or ethosomes. Ethosomes are phospholipid-based elastic nanovesicles containing a high content of ethanol. Ethanol is known as an efficient penetration enhancer and has been added in the vesicular system to prepare elastic nanovesicles. It can interact with the polar head group region of the lipid molecules, resulting in the reduction of the melting of the stratum corneum lipid, thereby increasing lipid fluidity and cell membrane permeability. Transfersomes possess an infrastructure consisting of hydrophobic and hydrophilic moieties together and as a result can accommodate drug molecules with wide range of solubility. Transfersomes can deform and pass through narrow constriction (from 5 to 10 times less than their own diameter) without measurable loss. Transferosomes were designed in an attempt to concentrate the drug in tissues of interest, while reducing amount of drug in the remaining tissues.

### Carriers

The composition of the invention comprises the dendrimer conjugate and a carrier. The term “carrier” or “vehicle” as used herein refers to carrier materials suitable for transdermal administration, and include any such materials known in the art, e.g., any liquid, gel, solvent, liquid diluent, solubilizer, hydrogel, paraffin, wax, oil, silicone, ester, oily cream, aqueous cream, water soluble base, glycerol, glycol, lotion , powder or microemulsion, polymer or the like, which is nontoxic and which does not significantly interact with other components of the composition or the skin in a deleterious manner. The carrier is present in an amount sufficient to achieve its function of carrying the dendrimer conjugate. Preferably, the carrier is present in an amount ranging from 2 to 99 wt %, more preferably 30 to 90 wt %, even more preferably 40 to 80 wt %.

Particularly preferred carriers are flexible, finite compositions. The phrase “flexible, finite system” is intended to mean a solid form capable of conforming to the surface with which it comes into contact, and which is capable of maintaining the contact in such solid form so as to facilitate topical application without adverse physiological response, and without being appreciably decomposed by aqueous contact during administration to a patient. Particularly preferred flexible, finite systems are polymer carriers such as pressure-sensitive adhesive matrix type in which the dendrimer conjugate is dispersed directly in the pressure-sensitive adhesive or reservoir type carriers.

Illustrative examples of suitable adhesives as matrix type flexible, finite delivery systems include those described in U.S. Pat. Nos. 5,474,783, and 5,656. Other flexible, finite systems known in the art include films, plasters, dressings, and bandages, as well as multilayer delivery systems in which the parent drug/prodrug is solubilized or contained in one or more separate layers and reservoir-type delivery systems in which the dendrimer conjugate is solubilized or contained in a reservoir or depot separate from the adhesive which attaches directly to the skin or mucosa.

As noted above, particularly preferred carriers are pressure-sensitive adhesive flexible, finite carriers. These can include any viscoelastic material which adheres instantaneously to most substrates with the application of very slight pressure and remains permanently tacky. A polymer is a pressure-sensitive adhesive within the meaning of the term as used herein if it has the properties of a pressure-sensitive adhesive per se or functions as a pressure-sensitive adhesive by admixture with tackifiers, plasticizers or other additives. The term pressure-sensitive adhesive also includes mixtures of different polymers and mixtures of polymers, such as polyisobutylenes (PIB), of different molecular weights, wherein each resultant mixture is pressure-sensitive. Other useful rubber based pressure-sensitive adhesives include hydrocarbon polymers such as natural and synthetic polyisoprene, polybutylene and polyisobutylene, styrene/butadiene polymers styrene-isoprene-styrene block copolymers, hydrocarbon polymers such as butyl rubber, halogen-containing polymers such as polyacrylic-nitrile, polytetrafluoroethylene, polyvinylchloride, polyvinylidene chloride, and polychlorodiene, and other copolymers thereof.

Other useful pressure-sensitive adhesives (“PSA”) can include acrylic-based pressure-sensitive adhesives and silicone-based pressure-sensitive adhesives as described in U.S. Pat. Nos. 5,474,783, and 5,656,386. Suitable commercially available acrylic-based polymers can include adhesives are commercially available and include the polyacrylate adhesives sold under the trademarks Duro-Tak by National Starch and Chemical Corporation, Bridgewater, N.J., such as Duro-Tak 87-2194, Duro-Tak 87-2196, Duro-Tak 87-1197, 87-4194, 87-2510, 87-2097 and 87-2852. Other suitable acrylic-based adhesives are those sold under the trademarks Gelva-Multipolymer Solution (GMS) (Monsanto; St. Louis, Mo.), such as GMS 737, 788, 1151, 3087 and 7882.

Suitable silicone-based pressure-sensitive adhesives can include those described in Sobieski, et al., “Silicone Pressure Sensitive Adhesives,” Handbook of Pressure-Sensitive Adhesive Technology, 2nd ed., pp. 508-517 (D. Satas, ed.), Van Nostrand Reinhold, N.Y. (1989), incorporated by reference in its entirety. Other useful silicone-based pressure sensitive adhesives are described in the following U.S. patents: U.S. Pat. Nos. 4,591,622; 4,584,355; 4,585,836; and 4,655,767. Suitable silicone-based pressure-sensitive adhesives are commercially available and include the silicone adhesives sold under the trademarks BIO-PSA 7-4503, BIO-PSA 7-4603, BIO-PSA 7-4301, 7-4202, 7-4102, 7-4106, and BIO-PSA 7-4303 by Dow Corning Corporation, Medical Products, Midland, Mich.

The amount of the polymer carrier can range from 2 to 99 wt %, preferably, 30 to 90 wt %, even more preferably 40 to 80 wt %.

The pressure-sensitive adhesives can be blended to modulate the solubility of the drug in the carrier system such as described in the ‘783 patent. In a particularly preferred embodiment of the invention, the multiple polymer adhesive system comprises a pressure-sensitive adhesive blend of an acrylic-based polymer, a silicone-based polymer, and a soluble PVP (described below). The acrylic-based polymer and silicone-based polymer are preferably in a ratio by weight, respectively, from about 2:98 to about 96:4, more preferably from about 2:98 to about 90:10, and even more preferably about 2:98 to about 86:14. The amount of acrylic-based (also referred to broadly as a polyacrylate) polymer and silicone-based polymer (also referred to broadly as a polysiloxane) is adjusted so as to modify the saturation concentration of the parent drug/prodrug in the ternary multiple polymer adhesive system in order to affect the rate of delivery of the parent drug/prodrug from the system and through the skin. Other useful ranges include about 5-85% by weight of the acrylate-based polymer, 10-90% by weight of polyisobutylene and 5-95% by weight of silicone-based polymer.

In some embodiments, the invention can also include a plasticizer or tackifying agent is incorporated into the formulation to improve the adhesive characteristics of the pressure-sensitive adhesive composition. Such plasticizers or tackifying agents include: (1) aliphatic hydrocarbons; (2) mixed aliphatic and aromatic hydrocarbons; (3) aromatic hydrocarbons; (4) substituted aromatic hydrocarbons; (5) hydrogenated esters; (6) polyterpenes; and (7) hydrogenated wood rosins.

The tackifying agent employed is preferably compatible with the blend of polymers. In preferred embodiments, the tackifying agent is silicone fluid (e.g., 360 Medical Fluid, available from Dow Corning Corporation, Midland, Mich.) or mineral oil. Silicone fluid is useful for blends comprising polysiloxane as a major component. In other embodiments, where a synthetic rubber, for example, is a major component, mineral oil is a preferred tackifying agent.

For parent dendrimer conjugates which are not readily soluble in the polymer system, a co-solvent for the dendrimer conjugate and polymer can be added. Co-solvents, such as lecithin, retinal derivatives, tocopherol, dipropylene glycol, triacetin, propylene glycol, saturated and unsaturated fatty acids, mineral oil, silicone fluid, alcohols, butyl benzyl phthalate, and the like are useful in the practice of the instant invention depending on the solubility of the parent drug/prodrug in the multiple polymer adhesive system.

In addition, crystallization inhibiting agents can be included in the compositions of the invention. One known agent is polyvinylpyrrolidone (PVP), preferably soluble PVP as described in detail in U.S. Pat. No. 6,221,383. The term “polyvinylpyrrolidone,” or “PVP” refers to a polymer, either a homopolymer or copolymer, containing N-vinylpyrrolidone as the monomeric unit. Typical PVP polymers are homopolymeric PVPs and the copolymer vinyl acetate vinylpyrrolidone. The homopolymeric PVPs are known to the pharmaceutical industry under a variety of designations including Povidone, Polyvidone, Polyvidonum, Polyvidonum solubile, and Poly(l-vinyl-2-pyrrolidone). The copolymer vinyl acetate vinylpyrrolidone is known to the pharmaceutical industry as Copolyvidon, Copolyvidone, and Copolyvidonum. The term “soluble” when used with reference to PVP means that the polymer is soluble in water and generally is not substantially cross-linked, and has a molecular weight of less than about 2,000,000. The PVP usable with the present invention, preferably has a molecular weight of about 2,000 to 1,100,000, more preferably 5,000 to 100,000, and most preferably 7,000 to 54,000.

The amount and type of PVP required in the foregoing preferred embodiment will depend on the quantity and type of dendrimer conjugate and/or therapeutic agent in the adhesive, as well as the type of adhesive, but can be readily determined through routine experimentation. Typically, the PVP is present in an amount from about 1% to about 20% by weight, preferably from about 3% to about 15% by weight. However, the amount of PVP can be higher than 20% for example, up to 40%, depending on the particular parent drug/prodrug used and on the desired properties of the blend. One commercially useful PVP is sold under “Kollidon,” such as “Kollidon 10,” “Kollidon 17 PF,” “Kollidon 25,” “Kollidon 90,” “Kollidon 30,” and “VA 64” a trademark of BASF AG, Ludwigshafen, Germany. Another useful PVP is sold under Kollidon CL-M also a trademark of BASF AG.

The compositions of this invention may further be provided with various thickeners, fillers and other additives known for use with transdermal drug delivery systems. Where the composition tends to absorb water, for example, when lecithin is used as a co-solvent, hydrophilic substances are especially useful. One type of hydrophilic substance which has been successfully employed is clay. The addition of clay has been found to improve adhesiveness in transdermal formulations without reducing the rate of parent drug/prodrug delivery. Suitable clays include aluminum silicate clay, kaolinite, montmorillonite, atapulgite, illite, bentonite, halloysite and the like.

### Chemical Penetration Enhancers

Despite seemingly obvious advantages, local transdermal therapy (LTT) systems in cancer prevention and therapy are utilized infrequently, because of limited drug penetration through skin layers. The topmost layer of the skin, stratum corneum (SC) is a strong hydrophobic barrier to LTT and does not typically allow permeation of hydrophilic, active therapeutic molecules which have a molecular weight >500 Da. Chemical penetration enhancers (CPEs) facilitate drug delivery. Among others, oleic acid (OA) is known to be one of the most effective CPEs that can interact with intercellular lipids, thereby enhancing skin permeability 9Guillard Eur. J. Pharm. Sci. 36: 192-199, 2009). It has a direct fluidizing action on the alkyl chains and an indirect action on the polar head groups of the lipid bilayers, resulting in a more spacing lipid packing. However, many of CPEs are small molecules that cause significant skin toxicity and irritation. In contrast, polymeric penetration enhancers (PPEs) do not cause skin irritation but their large molecular size prevents them from penetrating deep into the skin. Thus, overcoming the skin barrier safely and effectively remains a challenge

The dendrimer conjugates or compositions of the invention can also contain agents known to accelerate the delivery of the therapeutic agent and/or dendrimer through the skin. These agents have been referred to as chemical penetration enhancer, skin-penetration enhancers, accelerants, adjuvants, and sorption promoters, and are collectively referred to herein as “chemical penetration enhancers (CPE)” and are described in U.S. Pat. No. 6,221,383. CPE used in the invention include fatty acids such as oleic and linoleic acids, fatty alcohols, fatty alcohol ethers, biologics, enzymes, amines, amides, complexing agents, ionic compounds, dimetyl sulfoxide, N-methyl pyrrolidone, solvents, azones and surfactants.

For example, CPEs include polyhydric alcohols such as dipropylene glycol, propylene glycol, and polyethylene glycol which enhance parent drug/prodrug solubility; oils such as olive oil, squalene, and lanolin; fatty ethers such as cetyl ether and oleyl ether; fatty acid esters such as isopropyl myristate which enhance parent drug/prodrug diffusibility; urea and urea derivatives such as allantoin which affect the ability of keratin to retain moisture; polar solvents such as dimethyldecylphosphoxide, methyloctylsulfoxide, dimethyllaurylamide, dodecylpyrrolidone, isosorbitol, dimethylacetonide, dimethylsulfoxide, decylmethylsulfoxide, and dimethylformamide which affect keratin permeability; salicylic acid which softens the keratin; amino acids which are penetration assistants; benzyl nicotinate which is a hair follicle opener; and higher molecular weight aliphatic surfactants such as lauryl sulfate salts which change the surface state of the skin and drugs administered. Other agents include ascorbic acid, panthenol, butylated hydroxytoluene, tocopherol, tocopheryl acetate, tocopheryl linoleate, propyl oleate, and isopropyl palmitate. Particularly preferred are combinations of polyhydric alcohols such as glycerine, dipropylene glycol, butylene glycol, propylene glycol and one or more of oleyl alcohol and oleic acid.

### Therapeutic Agents

The anti-cancer agents and chemopreventive agents that find use in the present invention are those that are amenable to incorporation into dendrimeric structures or are otherwise associated with dendrimer structures such that they can be delivered into a subject, tissue, or cell without loss of fidelity of its anti-cancer effect or cancer-preventing effect. For a more detailed description of cancer therapeutic agents such as a platinum complex, verapamil, podophyllotoxin, carboplatin, procarbazine, mechlorethamine, cyclophosphamide, camptothecin, ifosfamide, melphalan, chlorambucil, busulfan, nitrosourea, adriamycin, dactinomycin, daunorubicin, doxorubicin, bleomycin, plicamycin, mitomycin, etoposide (VP16), tamoxifen, endoxifen, idoxifen, raloxifene, finasteride, aspataxol, transplatinum, 5-fluorouracil, vincristine, vinblastine, methotrexate, gemcitabine and other similar anti-cancer agents, those of skill in the art are referred to any number of instructive manuals including, but not limited to, the Physician's Desk reference and to Goodman and Gilman's “Pharmaceutical Basis of Therapeutics” ninth edition, Eds. Hardman et al., 1996.

Chemoprevention is a pharmaceutical approach to intervention in order to arrest of reverse the process of carcinogenesis. Chemopreventive agents are therapeutic agents that lower the risk of cancer or slow its development by preventing tumor growth or reducing the risk of tumor growth . These agents are not used to treat cancer. Examples include tamoxifen, endoxifen, 4-hydroxytamoxifen, raloxifene, 4-hydroxyphenylretinamide (fenertinide), finasteride and other drugs that reduce the amount of dihydrotestosterone, aspirin, non-seroidal anti-inflammatory drugs (NSAIDS) such as selective COX-2 inhibitors such as celecoxib, selective estrogen receptor modulators (SERMS) such as LY35381-HCl, selective binder to retinoid X receptors (rexinoids) such as LG 100268 and targretin (LGD 1069), peroxisome proliferator-activated receptor γ (PPAR-γ) ligands such as GW 7845.

Additional therapeutic agents include without limitation analgesics and analgesic combinations, anesthetics, anorexics, α-adrenergic agonists, β-adrenergic agonists, α-adrenergic blockers, β-adrenergic blockers, anti-allergics, antiarthritics, antiasthmatic agents, antibiotics, anticholinergics, anticonvulsants, antidepressants, antihemophilics, antidiabetic agents, antidiarrheals, antifungals, antianginals, antigens, antihistamines, antihypertensives, anti-inflammatories, antimigraine preparations, antinauseants, antineoplastics, antiparkinsonism drugs, antiprotozoans, antiameics, anthelmintics, antipruritics, antipsychotics, antipyretics, antispasmodics, antivirals, anabolics, androgens,anti-androgens, anti-estrogens, antiacne drugs, calcium channel blockers, cardiovascular preparations, central nervous system stimulants, contraceptives, cough and cold preparations including decongestants, diuretics, enzyme inhibitors, enzymes, genetic material including DNA and RNA, growth factors, growth hormones, hormone inhibitors, hypnotics, immunoactive agents, immunosuppressive agents, mineralcorticoids, microbicides, muscle relaxants, ophthalmic agents, prolactin, prostaglandins parasympatholytics, peptides, peripheral and cerebral vasodilators, proteins, psychostimulants, receptor agonists, sedatives, sulfonamides, quinolones, spermicides and other contraceptives, steroids, sympathomimetics, tranquilizers, vaccines, vitamins, vasodilators, polypeptides, vasodilating agents including general coronary, viral vectors, small organic molecules, and combinations thereof.

Androgens include Androgens such as Boldenone, Fluoxymesterone, Mestanolone, Mesterolone, Methandrostenolone, 17-Methyltestosterone, 17.alpha.-Methyltestosterone 3-Cyclopentyl Enol Ether, Norethandrolone, Normethandrone, Oxandrolone, Oxymesterone, oxymetholone, Prasterone, Stanlolone, Stanozolol, Testosterone, Testosterone 17-Chloral Hemiacetal, Testosterone 17.beta.-Cypionate, Testosterone Enanthate, Testosterone Nicotinate, Testosterone Pheynylacetate, Testosterone Propionate and Tiomesterone.

Anti-androgens include Bifluranol, Cyoctol, Cyproterone, Delmadinone Acetate, Flutimide, Nilutamide and Oxendolone.

Antiacne drugs include Antiacne drugs such as Algestone Acetophenide, Azelaic Acid, Benzoyl Peroxide, Cyoctol, Cyproterone, Motretinide, Resorcinol, Retinoic Acid and Tetroquinone.

Antiestrogens include delmadinone acetate, ethamoxytriphetol, tamoxifen and toremifene.

Antiviral agents include nucleoside phosphonates and other nucleoside analogs, 5-amino-4-imidazolecarboxamide ribonucleotide (AICAR) analogs, glycolytic pathway inhibitors, anionic polymers, and the like, more specifically: antiherpes agents such as acyclovir, famciclovir, foscarnet, ganciclovir, idoxuridine, sorivudine, trifluridine, valacyclovir, and vidarabine; and other antiviral agents such as abacavir, adefovir, amantadine, amprenavir, cidofovir, delviridine, 2-deoxyglucose, dextran sulfate, didanosine, efavirenz, entecavir, indinavir, interferon alpha and PEGylated interferon, interferon alfacon-1, lamivudine, nelfinavir, nevirapine, ribavirin, rimantadine, ritonavir, saquinavir, squalamine, stavudine, telbivudine, tenofovir, tipranavir, valganciclovir, zalcitabine, zidovudine, zintevir, and mixtures thereof. Still other antiviral agents are glycerides, particularly monoglycerides, which have antiviral activity. One such agent is monolaurin, the monoglyceride of lauric acid.

Anti-inflammatory agents include corticosteroids, e.g., lower potency corticosteroids such as hydrocortisone, hydrocortisone-21-monoesters (e.g., hydrocortisone-21-acetate, hydrocortisone-21-butyrate, hydrocortisone-21-propionate, hydrocortisone-21-valerate, etc.), hydrocortisone-17,21-diesters (e.g., hydrocortisone-17,21-diacetate, hydrocortisone-17-acetate-21-butyrate, hydrocortisone-17,21-dibutyrate, etc.), alclometasone, dexamethasone, flumethasone, prednisolone, or methylprednisolone, or higher potency corticosteroids such as clobetasol propionate, betamethasone benzoate, betamethasone diproprionate, diflorasone diacetate, fluocinonide, mometasone furoate, triamcinolone acetonide, and mixtures thereof.

Antibiotic agents include those of the lincomycin family, such as lincomycin per se, clindamycin, and the 7-deoxy,7-chloro derivative of lincomycin (i.e., 7-chloro-6,7,8-trideoxy-6-[[(1-methyl-4-propyl-2-pyrrolidinyl)carbonyl]amino]-1-thio-L-threo-alpha-D-galacto-octopyranoside); other macrolide, aminoglycoside, and glycopeptide antibiotics such as erythromycin, clarithromycin, azithromycin, streptomycin, gentamicin, tobramycin, amikacin, neomycin, vancomycin, and teicoplanin; antibiotics of the tetracycline family, including tetracycline per se, chlortetracycline, oxytetracycline, demeclocycline, rolitetracycline, methacycline and doxycycline; and sulfur-based antibiotics, such as the sulfonamides sulfacetamide, sulfabenzamide, sulfadiazine, sulfadoxine, sulfamerazine, sulfamethazine, sulfamethizole, and sulfamethoxazole; streptogramin antibiotics such as quinupristin and dalfopristin; and quinolone antibiotics such as ciprofloxacin, nalidixic acid, ofloxacin, and mixtures thereof.

Antifungal agents include miconazole, terconazole, isoconazole, itraconazole, fenticonazole, fluconazole, ketoconazole, clotrimazole, butoconazole, econazole, metronidazole, 5-fluorouracil, amphotericin B, and mixtures thereof.

Antihemophilic agents include antifibrinolytic amino acids, aprotinin, 1-deamino-8-d-arginine vasopressin, aminocaproic acid, tranexamic acid and conjugated estrogens, and mixtures thereof (Mannucci et al. (1998). New. Eng. J. Med. 339:245)

Other anti-infective agents include miscellaneous antibacterial agents such as chloramphenicol, spectinomycin, polymyxin B (colistin), and bacitracin, anti-mycobacterials such as such as isoniazid, rifampin, rifabutin, ethambutol, pyrazinamide, ethionamide, aminosalicylic acid, and cycloserine, and antihelminthic agents such as albendazole, oxfendazole, thiabendazole, and mixtures thereof.

Any of the therapeutic agents may be administered in the form of a salt, ester, amide, prodrug, conjugate, active metabolite, isomer, fragment, analog, or the like, provided that the salt, ester, amide, prodrug, conjugate, active metabolite, isomer, fragment, or analog is pharmaceutically acceptable and pharmacologically active in the present context. Salts, esters, amides, prodrugs, conjugates, active metabolites, isomers, fragments, and analogs of the agents may be prepared using standard procedures known to those skilled in the art of synthetic organic chemistry and described, for example, by J. March, Advanced Organic Chemistry: Reactions, Mechanisms and Structure, 5th Edition (New York: Wiley-InterScience, 2001).

Pharmaceutically acceptable salts include the acid addition salts (formed with the free amino groups of the peptide) and which are formed with inorganic acids such as, e.g., hydrochloric or phosphoric acids, or such organic acids as acetic, oxalic, tartaric, mandelic. Salts formed with the free carboxyl groups may also be derived from inorganic bases such as, e.g., sodium, potassium, ammonium, calcium, or ferric hydroxides, and such organic bases as isopropylamine, trimethylamine, 2-ethylamino ethanol, histidine, and procaine.

### Tamoxifen and Endoxifen

In 1998, the results of the NSABP P01 trial demonstrated the ability to reduce breast cancer risk by about 50% with the triphenylethelylene (TPE), tamoxifen (TAM), taken orally for five years. This finding appeared to herald a new era of successful chemoprevention of breast cancer, but the promise has not been realized. Tamoxifen is poorly accepted by the estimated 1 million women in the United States who are risk-eligible for breast cancer chemoprevention, and another 40,000 women who are diagnosed with ductal carcinoma in situ (DCIS) each year. For these women, TAM is prescribed only for its local effects on the breasts (i.e. systemic therapy is not required). It is a pro-drug which is efficiently absorbed following oral administration, and is metabolized to its active forms: 4-hydroxytamoxifen (4-0HT) and endoxifen (ENX). TAM reduces the risk of DCIS recurrence and prevents new primary tumors of the breast. The side-effects of TAM which lead many women to decline therapy include thrombo-embolism, uterine cancer, and quality of life impairment through hot flashes and vaginal atrophy. Safe, widely accepted and effective alternatives for breast cancer prevention are thus badly needed. An alternative approach with the potential of reducing or eliminating systemic toxicity is that of local, transdermal application of active drugs. To be clinically successful, topically applied agents must penetrate the skin, distribute throughout the entire breast, and reach effective concentrations in the target cells. Moreover, systemic levels of these agents must be substantially lower than when these agents are taken orally. Results from a small Phase II study (Rouanet et al. J. Clin. Oncol. 23: 2980-2987, 2005) demonstrate that an active metabolite of TAM, 4-hydroxytamoxifen (4-0HT), applied to the breast as a gel, may meet these conditions.

Existing data regarding transdermal delivery of 4-OHT: The monohydroxy metabolite of TAM, 4-0HT, has a 25 to 50-fold greater affinity for the estrogen receptor a (ERa) than TAM. It suppresses breast cancer cell growth in tissue culture with two-log greater potency, and has been shown to inhibit the growth of normal human breast cells, but has little activity when delivered orally due to its rapid conjugation and inactivation by the liver. In 1986, Mauvais-Jarvis et. al. showed the feasibility of transdermal administration of 4-0HT in a study where trans-[311]-4-0HT (9 women) and trans-[³H]TAM (3 women) was applied to the skin of the breast. Tumor, blood and urine samples obtained prior to, and 12 hours to 7 days following application showed significantly greater retention of 4-0HT in breast tissue than TAM (Mauvais-Javis et al., Can. Res. 46: 1521-1525, 1986). Subsequently, in a small pre-surgical Phase II trial, post-menopausal women with ER positive invasive breast cancer were randomized to a range of concentrations of 4-0HT gel applied to the skin of the breast, or oral tamoxifen at 20 mg daily. The primary endpoint was a reduction in tumor cell proliferation measured by Ki-67 labeling. A 4-0HT dose of 0.5 or 1 mg per breast per day reduced the Ki-67 labeling to a similar degree as oral TAM. Resultant plasma levels of 4-OHT were 0.05 and 0.13 ng/mL, respectively, and were thus approximately 1/30 to 1/12, respectively, of those seen after 20 mg of oral TAM (Rouanet et al. J. Clin. Oncol. 23: 2980-2987, 2005). Other studies have shown that the concentration of 4-0HT in the breast tissue is higher if the drug is applied to the skin of the breast than to the skin of the abdomen or arm (Mauvais-Javis et al., Can. Res. 46: 1521-1525, 1986). There is an anatomic rationale for this since the mammary gland is a skin appendage and lymphatic flow of the mammary skin and the parenchyma has similar drainage. However, less than 1% of the drug applied to the skin in gel form is found in mammary parenchyma, and concentrations of active metabolites achieved in the breast with local transdermal therapy (LTT) are significantly lower than those attained with oral TAM 1 mg daily, arid may be insufficient for therapeutic effect, leaving considerable room for improvement.

Additionally, there are potential compliance barriers to the use of the present hydroalcoholic gel formulation of 4-0HT (Besins Healthcare, Paris, France): it needs to be applied daily after a shower, the skin cannot be washed for 6-8 hours after application, interfering with activities such as swimming or bathing; and sun exposure is discouraged. Formulations that achieve higher concentrations, with prolonged retention, and require less frequent application may therefore be more effective.

Endoxifen (ENX), the other major active TAM metabolite has similar efficacy to 4-OHT in modulating ER expression, and is the more important metabolite in women taking oral TAM because of its greater abundance. Women with CYP2D6 polymornhisms metabolize TAM poorly, have low plasma concentrations of ENX. ENX has potential advantages over 4-OHT; it causes proteosomic degradation of ERa , whereas 4-OHT stabilizes ERa 21. ENX also has a secondary amine group that is more reactive towards carboxylic acid or anhydride than the tertiary amine on 4-OHT, providing more options to enhance the efficiency of transdermal delivery through covalent conjugation with penetration enhancers.

The dendrimer conjugates of the present invention provide a transdermal delivery system that that will reduce the systemic exposure of chemopreventive agents such as TAM and ENX and thereby reduce the adverse effects of the agent on the subject. The ease of administering the transdermal compositions comprising the dendrimer conjugate associated with a chemopreventive agent, such as TAM or ENX, will increase patient compliance and will have better results in preventing tumor formation and reducing the risk for developing a primary or secondary tumor.

### EXAMPLES

### Example 1

### Generation of Dendrimer Constructs

### Materials

PAMAM dendrimers, generations 2 (G2, MW 3256 g/114 mol) and 4 (G4, MW 14 215 g/mol), with ethylenediamine cores were purchased from Sigma-Aldrich (St. Louis, Mo.). Rhodamine B isothiocyanate (RITC), acetic anhydride, triethylamine (TEA), succinic anhydride, oleic (OA), N-(3-dimethylaminopropyl)-N′-ethylcarbodiimide hydrochloride (EDC), N-hydroxysuccinimide (NHS), anhydrous methanol, ethanol, dimethyl sulfoxide (DMSO), and 1-octanol were all obtained from Sigma-Aldrich (St. Louis, Mo.). Calcium- and magnesium-free PBS was purchased from Mediatech. (Manassas, Va.). Polyethylene glycol 400 (PEG400) was obtained from Fisher Scientific, (Fair Lawn, N.J.). All other chemicals used in this study were obtained from Sigma-Aldrich and used as received unless otherwise noted.

### Synthesis and Characterization of G2- and G4-RITC-NH₂ Conjugates

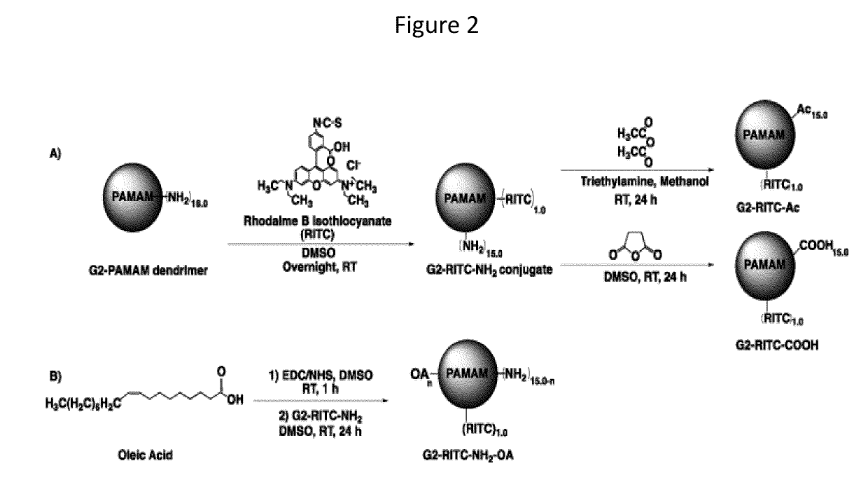

The reaction scheme for conjugation between G2 and £2 128 G4 PAMAM dendrimers and RITC is illustrated in FIG. 2A. The reactions were carried out following previous reports (Filipowicz et al., Intl. J. Pharma. 408: 152-156, 2011; Majoros et al., J. med. Chem., 48(19):5892-5899, 2005). In brief, G2 and G4 PAMAM dendrimers (10.0 mg, 3.1 μmol and 0.7 μmol, respectively) were dissolved in 1 mL of DMSO. RITC (2.5 mg, 4.6 132 μmol and 0.6 mg, 1.1 μmol, 50% molar excess to G2 and G4, respectively) was first dissolved in 200 μL of DMSO and then added to the dendrimer solutions drop wise under vigorous stirring at room temperature for 24 hours resulting in G2-RITC-NH₂ and 41-RITC-NH₂, respectively. Unreacted RITC was removed by membrane dialysis (Spectra/Port dialysis membrane, MWCO of 500 for G2 and 1000 for G4, Spectrum Laboratories, Rancho Dominguez, Calif.) in 4 L of double-deionized water (ddH20) for 3 days. The purified products were then lyophilized for 2 days and stored at −20° C. The chemical structure of the conjugates was confirmed by ¹H NMR. in D₂0 using a 400 MHz Bruker DPX-400 spectrometer (Bruker BioSpin, Billerica, Mass.).21 The numbers of the RITC molecules per dendrimer were calculated from UV/vis measurements. Serially diluted RITC (1.3, 2.5, 5.0, 10.0 μg/145 mL) solutions in 1:1 DMSO/H₂0 were prepared and used to plot the standard curve for the quantification of the number of RITC attached to the dendrimer conjugates using a DU800 UV/vis spectrophotometer (Beckman Coulter, Calif.). The surface charges (zeta potential, millivolts) of G2-RITC-NH₂ and G4-RITC-NH₂ were obtained from three repeat measurements of the aqueous dendrimer solutions at a concentration of 125 μg/ml by quasi-elastic laser light scattering using a Nicomp Zeta Potential/Particle Sizer (Particle Sizing Systems, Santa Barbara, Calif.), as previously reported (Bae et al., Chem Commun. 47(37):10302-10304, 2011; Sunoquot et al., Bioconjugate Chem. 22(3): 466-474).

### Preparation and Characterization of Acetylated and Carboxylated G2-RITC Conjugates.

G2-RITC-NH₂ was fully acetylated or carboxylated as previously described (Hong et al., Bioconjugate Chem. 15(4): 774-782, 2004; , Hong et al., Bioconjugate Chem. 20(8): 1503-1513, 2009) In brief, G2-RITC-NH₂ (10.0 mg, 2.6 μmoL) was dissolved in 1 mL of methanol and 158 acetylated by adding acetic anhydride (6.1 mg, 5.6 μL, 59.3 μmoL, 50% molar excess of the number of amines on the surface of G2-RITC-NH2) and TEA (6.7 mg, 9.2 μL, 65.2 μmol, 10% molar excess of acetic anhydride), under vigorous stirring at room temperature for 24 hours (FIG. 2A). In a separate reaction, G2-RITC-NH₂ (10.0 mg, 2.6 μmoL) in 1 mL of DMSO was fully carboxylated by adding succinic anhydride (5.9 mg, 59.3 μmoL, 50% molar excess of the number of amines on the surface of G2-RITC-NH₂) in 1 mL of DMSO under vigorous stirring at room temperature for 24 hours (FIG. 2A). The acetylation and carboxylation reactions were confirmed using ¹H NMR measurements. The surface charges (zeta potential, 169 millivolts) of G2-RITC-Ac, G2-RITC-COOH, and G2-RITC-NH₂ were also obtained using the same method as described above.

### Preparation and Characterization of G2-RITC-NH2-0A Conjugates.

G2-RITC-NH₂ was reacted with OA at either 5 or 8 molar excess to G2-RITC-NH₂ using EDC/NHS chemistry (FIG. 2B). For the stoichiometry of 1:5 of G2-RITC-NH₂/OA, OA (4.2 μL, 13.2 175 μmol) was preactivated by EDC (25.3 mg, 132.0 μmol) and NHS (15.2 mg, 131.9 μmol) in DMSO with vigorous stirring in the dark at in room temperature for 2 hours. G2-RITC-NH₂ (10.0 mg, 2.6 μmol) in DMSO was then added and vigorously stirred for 24 hours. For the 1:8 ratio, proportionally higher amounts of OA, EDC, and NHS were used under the identical conditions. Unreacted OA was removed by membrane dialysis against ddH₂O using a 500 MWCO membrane (Spectrum Laboratories) for 2 days, followed by lyophilization for 2 days and storage at −20° C. ¹H NMR and mass spectroscopy (MS, Applied Biosystems Voyager-DE 184 Pro matrix-assisted laser desorption ionization time-of-flight (MALDI- 185 TOF) mass spectrometer, Carlsbad, Calif.) were performed to characterize the molecular weights of the dendrimer-OA conjugates 187 as previously described (Bae et al., Chem Commun. 47(37):10302-10304, 2011; Lalwani et al. Bioconjugate Chem. 22(3): 466-474, 2011).

Statistical Analysis. Data processing was performed using Origin 302 8.0. Statistical analysis was performed using SPSS 11.5 based on a one-way ANOVA at p<0.05.

### Example 2

Characterization of Various Dendrimer Conjugates Comprising RITC

The molecular weights and numbers of the terminal groups of each dendrimer as well as the numbers of fluorophores (RITC) and OA (oleic acid) attached to each dendrimer are summarized in Table 1.

TABLE 1
Characterization of Dendrimer Conjugates

| | surface | fluorophore | | | | |
|---|---|---|---|---|---|---|
| | NH₂ groups | attached′ | fluorophore attached | measured MW (Da)′ | theoretical MW (Da) | c-potential (mV)^{a} |
| G2-NH₂ | 16 | 0 | 0 | 3160-3253 | 3256 | +16.6 |
| G4-NH₂ | 64 | 0 | 0 | 10 709-15 287 | 14 215 | +38.5 |
| G4-RITC-NH₂ | 63 | 1.0 | | 9101-19 000 | 15 753 | +41.2 |
| G2-RITC-NH₂ | 15 | 1.0 | 1.3 | 4155 | 3792 | +18.8 |
| G2-RITC-Ac | 0 | 1.0 | 1.3 | 4399 | 4398 | +2.7 |
| G2-RITC-COOH | 0 | 1.0 | 1.3 | 5655 | 5346 | −14.5 |
| G2-RITC-NH₂-0A₂₃ | 13 | 1.0 | 1.3 | 4389 | 4638 | N/A′ |
| G2-RITC-NH₂-0A₂₃ | 10 | 1.0 | 1.3 | 3708-5643 | 5202 | N/A^{t} |

The UV/vis measurements revealed that, on average, 1.0 RITC was conjugated to each G2 and G4 dendrimer molecule, which were referred to as G2-RITC-NH₂ and G4-RITC-NH₂, respectively. The RITC conjugation was confirmed using ¹H NMR by observing the peak(s) from the newly formed thiourea bond at 6.96 ppm as a result of conjugation between the dendrimers and the isothiocyanates. The degree of acetylation on the surface of the G@-RITC-NH₂ dendrimers was measured using ¹H NMR, which revealed that 94% of the primary amine groups on the G2 surfaces were 322 converted to acetamide. The shape changes of the characteristic peaks of the dendrimers after carboxylation demonstrated that the surface modification was successful. The zeta potential values of the various dendrimers are also listed in Table 1. G4-RITC-NH₂ exhibited the highest positively charged moiety (+41.2 mV), and G2-RITC-NH2, G2-RITC-Ac, and G2-RITC-COOH showed highly positive (+18.8 mV), nearly neutral (+2.7 330 mV), and negative (-14.5 mV) surface charges, respectively, confirming the success of the surface modifications. The reaction stoichiometry (1:5 and 1:8 of G2-RITC-NH₂:OA) resulted in two different numbers of OA molecules per dendrimer, as shown in Table 1.

The NMR and MALDI-TOF data indicated that 2.3 and 2.7 OA molecules were conjugated per G2-RITC-NH₂ molecules, resulting in G2-RITC-NH2-OA_{2.3} and G2-RITC-NH2-OA_{2.7}, respectively.

### Example 3

### Effect of Dendrimer Size on Skin Permeation

It is generally known that the smaller molecules penetrate through the skin layers more efficiently than their larger counterparts. However, it is difficult to compare the size effect in polymeric materials while maintaining other parameters constant due to their intrinsic heterogeneity in structure and chain length. Dendrimers offer precise control over their size, providing an excellent platform for systematic studies to investigate the effects of not only size but also other parameters. We therefore compared the skin permeation G2 and G4 PAMAM dendrimers conjugated with RITC to investigate the size effect.

In order to determine the effect of dendrimer size on skin permeation, Franz Diffusion Cells were used with porcine skin. Full-thickness porcine skin was collected from the inner thigh area of a 30 lb female American 190 Yorkshire pig (Halsted Packing House, Chicago, Ill.). The skin samples were collected from the thigh regions because these areas have generally less hair and fat compared with other regions such as dorsal, flank, and belly, to minimize the hair and fat removal process that may cause skin damage. All hairs were removed using small tweezers, and the skin was carefully examined for any defects. Undamaged skin was 196 cut into 10×10 cm² squares with similar numbers of hair follicles. The fat and subcutaneous tissues were gently removed using a surgical blade. Each piece of skin was wrapped with aluminum foil, sealed in a zip bag, stored at −80° C., and used within 90 days.

The porcine skin was thawed on ice, further trimmed into 1.2×1.2 cm² squares, and sandwiched between the donor and receiver chambers of the Franz diffusion cells (φ7 mm with 0.38 cm² exposure area, PermeGear, Hellertown, Pa.) with the SC side facing upward, following a previous report (Tong et al., Mol. Pharm 10(10): 1235-1347). The receiver chambers were then filled with fresh PBS (pH 7.4). After equilibration of the skin at 37° C. for 30 minutes, 100 gL of each dendrimer conjugate at a concentration of 1 mM or control groups (free rhodamine or vehicle (ddH20)) was applied to each donor chamber, as described in Venuganti et al., Intl. J. Pharma. 361(1-2):230-238, 2008. Note that for the dendrimer-OA conjugates, 70% ethanol solution was used as a solvent vehicle due to their poor water solubility. The chambers were first covered by Parafilm to prevent evaporation and then by aluminum foil to minimize fast photobleaching of rhodamine. The first sampling (t =0) was done by withdrawing 250 μL of receiver solution from each sampling portal, followed by the addition of 250 μL of fresh PBS to maintain a constant total volume in the receiver chamber. Samplings were performed as frequently as every 2 hours up to 24 hours. All sample solutions were kept at 4° C. in the dark before subsequent analysis.

The fluorescence intensity from each receiver solution was detected using a SpectraMAX GeminiXS microplate spectrofluorometer (Molecular Devices, Sunnyvale, Calif.). The dendrimer-RITC conjugates were detected at 555 nm excitation and 590 nm emission wavelengths. The amount of dendrimer in the receiver solutions was quantified based on standard curves of fluorescence intensities versus concentrations of serially diluted solutions from the 1 mM stock solutions of the various dendrimer conjugates (Table 1). The percent permeation (% Permeation) was calculated by dividing the amount of the conjugates in the receiver solution by the original amount applied. The materials absorbed to the epidermis and dermal layers were directly measured by extracting the conjugates from the each skin layer using a cocktail of 1:1:1 ddH₂O/ethanol/PEG400 for 6 hours. In some experiments, skin absorption was also measured by subtracting the amount of the materials in the donor and receiver solutions from the total amount of the materials applied.

To confirm that the skin used was intact, we performed two observations of each square of skin were performed before accepting the data it generated. First, because the dendrimer-RITC conjugates were red in color, we observed immediate color changes (within 1 h) in receiver solutions when damaged skin was used, and such results were excluded. Second, we measured the fluorescence from the receiver solutions using a fluorimeter at early time points (0, 2, and 4 h) to ensure that there was the induction period that is typical for skin permeation. Early permeation was considered to be an indicator for skin damage.

For the size comparison study, porcine skin was exposed to either G2-RITC-NH₂ or G4-RITC-NH₂ for 24 hours in the Franz cell setup. The skin area that was exposed to the treatment was carefully collected, rinsed twice with ddH₂O for 10 minutes, and immersed in 10 mL of 10% neutral buffered formalin for 24 hours of fixation. The fixed skin pieces were transferred into 70% (v/v) ethanol for 24 hours of dehydration, followed by an overnight treatment of 30% (w/v) sucrose solution, before being embedded into cryomolds (Tissue-Tek Sakura Finetek USA, Torrance, Calif.). Skin was cryosectioned into 10 mm thick slices and placed on antifrost glass slides. After drying at room temperature, the slides were first stained with Wheat Germ Agglutinin-Alexa Fluor 488 conjugate (WGA-AF488, 5 μg/mL in PBS, Invitrogen, Carlsbad, Calif.) for 10 minutes at room temperature. After washing off the excess WGA-AF488 with ddH₂O, skin slides were mounted with antiphotobleaching mounting media with DAPI (Vector Laboratory, Burlingame, Calif.) and covered with glass coverslips.

For the permeation pathway study, porcine skin was cut into 5 x 10 mm² strips and embedded into cryomolds. Skin was cryosectioned into 10 mm² thick slices and placed on antifrost glass slides. After drying at room temperature, the skin strips were treated with 200 nM of G2-RITC-NH₂, G2-RITC-COOH, or G2-RITC-Ac in PBS at RT for 1 hour, and the excess materials were gently washed away using ddH₂O. The slides were then fixed by 10% neutral buffered formalin for 10 minutes at room temperature and washed again with ddH₂O. The slides were stained with WGA-AF488 for 10 minutes at RT. After washing off the excess WGA-AF488 with ddH₂O, the skin slides were mounted with antiphotobleaching mounting media with DAPI and covered with glass coverslips.

The cross sections of the skin layers were then visualized using a Zeiss LSM 510 Meta confocal laser scanning microscope (CLSM, Carl Zeiss, Germany). The 488 nm line of a 30 mW tunable argon laser was used for the excitation of AF488, a 1 mW HeNe at 543 nm for RITC, and a 25 mW diode UV 405 nm laser for DAPL Emission was filtered at 505-530, 565-595, and 420 nm for AF488, RITC, and DAPI, respectively.

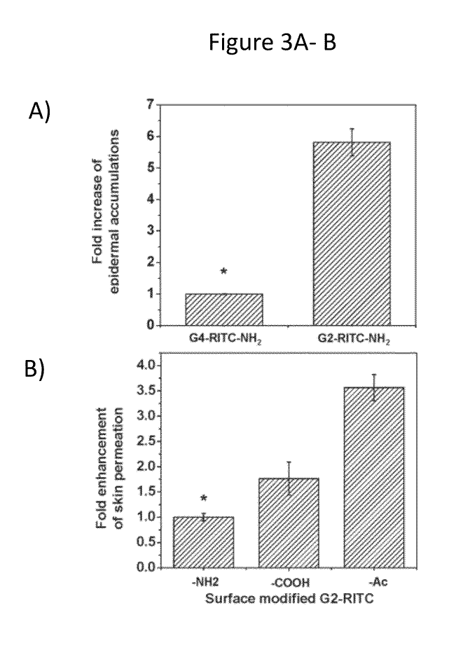

The effect of dendrimer size was investigated by comparing G2 and G4 PAMAM dendrimers conjugated with RITC. The confocal images of the cross sections of the porcine skin treated with G2-RITC-NH₂ and G4-RITC-NH₂. The images demonstrated that G4-RITC-NH₂ did not penetrate across the SC because only a small amount of the dendrimers was retained at the outermost layer of S. A significantly larger amount of G2-RITC-NH₂ was absorbed into the SC layers as well as the underlying viable epidermis G2-RITC-NH₂also exhibited a significantly stronger fluorescence signal than that of G4-RITC-NH₂, indicating that G2 dendrimers were much more strongly absorbed into the skin layers than G4. The %permeations of dendrimers showed better permeation of G2-RITC-NH2 than G4-RITC-NH2 in general (data not shown). However, the small amount of the permeated dendrimers and the large batch-by-batch variations made accurate quantification of the amount of dendrimers in the receiver solution difficult. Therefore, the amounts of dendrimers in the skin layers were measured instead of measuring % permeation. To quantify the dendrimer conjugates in the skin, the skin samples were collected after the Franz cell diffusion experiments, and the conjugates were extracted using a cocktail of 1:1:1 ddH₂O/ethanol/PEG400 for 6 hours. FIG. 3A demonstrates the fold increases of the skin-absorbed materials after 24 hours. The epidermal absorption of G2-RITC-NH₂ was 5.8 times higher than that of G4-RITC-NH₂ after 24 hours, indicating that the G2 dendrimer penetrates more efficiently than G4. Because of the better permeation of the smaller-sized G2 dendrimers, these dendrimers were used for subsequent transdermal permeation experiments that investigate the effect of charges (surface groups) and hydrophobicity on dendrimer skin interactions.

The Franz diffusion cell experiments revealed that the G2 conjugates displayed better skin permeation properties (up to 3.5% and up to 0.6% for G2 and G4 after 24 hours, respectively), which is in a good agreement with literature. Furthermore, the confocal images of skin cross-section demonstrated that G2 conjugates penetrate deeper into the skin layers compared with G4 PAMAM dendrimers, validating the first hypothesis. Although the dendrimer conjugates used in this study underwent an extensive purification process, it needs to be noted that the conjugates may contain a degree of larger impurities, such as dimers, which may further prevent the skin penetration of particularly larger (G4) dendrimers.

Any molecule larger than 500 g/mol is generally considered impermeable through the skin.° However, G2-RITC-NH₂, which has a molecular weight of as high as 5000 g/mol, still exhibits a degree of permeability and deep penetration through the porcine skin, which implies that it utilizes an alternative mechanism of penetration. Venuganti and Perumal also found, through transepidermal water loss, skin resistance measurements, and ATR-FTIR studies, that cationic dendrimers alter the skin lipid layers. G2 PAMAM dendrimers reduced skin resistance to a greater extent than higher generations of dendrimers (Venuganti et al.,Intl. J. Pharma. 361(1-2): 230-238, 2008). Moreover, a series of papers published by Hong et al. demonstrated that cationic PAMAM dendrimers, such as primary amine-terminated ones, induced nanoscale holes on supported lipid bilayers (Leroueil e t al., Acc. Chem. Res. 40(5): 335-342, 2007; Hong et al. Bioconjugate Chem., 1793: 728-734, 2009; Hong et al., Bioconjugate Chem. 20(8): 1503-1513, 2009). This membrane permeabilization mechanism plays a key role in the cellular internalization of PAMAM dendrimers and other positively charged polymers that have been commonly used for nonviral cell transfection or gene delivery (Hong et al., Bioconjugate Chem. 20(8): 1503-1513, 2009). The reduced skin resistance and membrane permeabilization by positively charged dendrimers may explain the observed skin permeation/penetration of the materials.

These experiments were substantiated with G2 and G4 dendrimers conjugated with fluorescein isothiocyanate (FITC) denoted as G2-FITC-NH₂ and G4-FITC-NH₂. The results of this experiment are summarized in Table 2. 3.52% (±1.16) G2-FITC (n=3) permeated through intact mouse skin layers over 24 hours, while 0.52% (±0.21) G4-FITC (n=2) permeated into the receiver solution. There was a 5.92-fold increase of skin permeation of G2-FITC verses G4-FITC.

TABLE 2

| | | | | Amount |
|---|---|---|---|---|
| | | Functional Groups, | FITC | Applied each |
| Groups | Concentration | (—NH2) | concentration | Franz Cell |
| G2-(FITC)₂ | 1.0 mM | (16-2) * 1.0 = 14 | 2 * 1.0 = 2 mM | 200 μL |
| G4-(FITC)₄ | 0.5 mM | (32-4) * 0.5 = 14 | 4 * 0.5 = 2 mM | 200 μL |
| Vehicle (H₂0) | / | / | / | 200 μL |

### Example 4

### Effect of the Surface Charge of Dendrimers on Skin Permeation and Retention

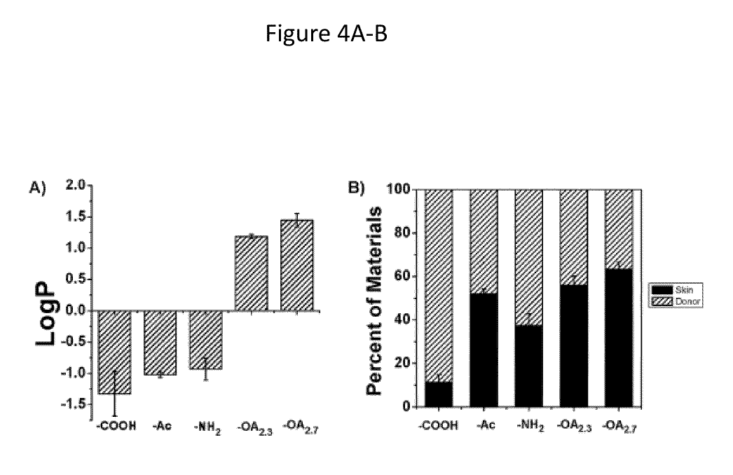

To assess the permeation efficiencies of the G2-RITC conjugates with different surface functional groups, Franz diffusion cell experiments were performed using a 1 Mm concentration of the materials in ddH₂O without adding any commonly used permeation enhancers. The difference in permeation was observed by analyzing RITC fluorescence in the receiver solutions (FIG. 3B). Although % permeation of the dendrimer conjugates was low (less than 3%), there was an approximately 4-fold increase in permeation for both G2-RITC-COOH and G2-RITC-Ac compared with G2-RITC-NH₂. These results show that G2 dendrimers with different surface functional groups behave differently in terms of skin permeation. Confocal images shown in FIG. 4 visualize the interactions of the various dendrimers within the skin cells, assessed by incubation of the precryosectioned skin slides with the dendrimer conjugates. For clear visualization of the dendrimers in the skin layers, the dendrimer conjugates were added after cryosectioning the skin samples. G2-RITC-NH₂ was internalized into the individual cells in both the epidermal and dermal layers. By way of contrast, neither G2-RITC-COOH nor G2-RITC-Ac interacted with the cells.

The skin permeation tests and confocal microscopy observations on cross sections of the porcine skin demonstrated that G2-RITC-NH₂ permeated the skin less effectively that G2-RITC-COOH and G2-RITC-AC (FIG. 3B). Interestingly, when delivering 5FU through the skin pretreated with PAMAM dendrimers with different surface functional groups, it was reported that the order of enhancement in Kₚ of 5FU was G4-NH₂>G4-OH>G3.5-COOH (Venugantiet al., J. Pharma. Sci. 361: 95(1520-3017):1345-2356, 2008). However, the permeation behaviors of surface-modified dendrimers themselves do not necessarily parallel the permeation enhancement effects for a small molecule. As previously reported, amine-terminated PAMAM dendrimers internalized into the cells nonselectively (Hong et al. Bioconjugate Chem., 17(3): 728-734, 2009). The negatively charged dendrimers did not internalize or bind to the cells. Because of the concentration gradient across the skin and possibly charge repulsions between the dendrimers and the negatively charged cell membrane, it is hypothesized that they go through the skin layers through an extracellular pathway that could be faster than the transcellular pathway taken by G2-RITC-NH₂. In contrast, the surface of G2-RITC-Ac is nearly neutral. It may simply follow the concentration gradient to go through the skin layers extracellularly, which also may result in a faster penetration compared with G2-RITC-NH₂. Because the theoretical molecular weight of G2-RITC-Ac (4398 g/mol) is smaller than G2-RITC-COOH (5346 g/mol), and based on the conclusion from the first hypothesis, it may go through the skin layers faster than G2-RITC-COOH. Because of the small size and flexible/deformable nature of PAMAM dendrimers even after surface modification, they may go through the skin layers more easily by taking the extracellular route, which results in the higher permeation efficiencies observed with G2-RITC- COOH and G2-RITC-Ac compared with G2-RITC-NH₂.

This hypothesis was further tested by confocal microscopy observations. G2-RITC-NH₂ internalized into the skin cells within 1 hour. This could explain the lower skin permeation efficiency of G2-RITC-NH₂ compared with G2-RITC-COOH and G2-RITC-Ac, which did not internalize into the cells. Amine-terminated dendrimers internalized into individual cells both in the epidermal and dermal layers by interactions between their positively charged termini and the negatively charged cell membranes (Hong et al. Bioconjugate Chem., 17(3): 728-734, 2009). This increased uptake leads to higher accumulation of the materials in the skin layers, which makes them potential candidates for localized treatment of skin diseases. Meanwhile, carboxylated and acetylated dendrimers appeared to penetrate the skin layers better than their amine-terminated counterparts (FIG. 3B). Charge repulsions, particularly between the carboxylated dendrimers and the cell membranes, may have forced them to take an extracellular route, allowing this rapid diffusion. This result highlights the potential advantage of using the carboxylated (or acetylated) dendrimers for systemic administration of active compounds through the skin, which requires fast and deep penetration through the skin layers and access to the circulation.

### Example 5

### Effect of Dendrimer Hydrophobicity on Skin Permeation

The partition coefficients of the surface-modified G2-RITC and the dendrimer-OA conjugates were determined using the shake-flask method (Dinerman et al. Macromol. Biosci. 10(10): 1235-1247, 2010. Equal volumes of spectroscopic grade 1-octanol and calcium- and magnesium-free PBS were stirred together vigorously for 24 hours to saturate the two phases mutually. The phases were allowed to separate overnight before aliquots were collected. Each of the water-soluble dendrimer conjugates (except for G2-RITC-NH₂-OA) was dissolved at a concentration of 10 μM in PBS. The G2-RITC-NH₂-OA conjugates were dissolved at 10 μM in 1-octanol The pH of each PBS solution was adjusted to 7.4 with NaOH, HCl, or both. To a 7 mL centrifuge tube was gently added 2 mL of the PBS phase and 2 mL of the octanol phase. The tube was placed on a rocker (Fisher Scientific, Pittsburgh, Pa.) rotating once every 3 seconds for 5 minutes and centrifuged at 20-60×g for 3 minutes. The fluorescence of the PBS phase was read using a SpectraMAX GeminiXS microplate spectrofluorometer (Molecular 297 Devices, Sunnyvale, Calif.) at 555 nm excitation and 590 nm emission wavelengths. The partition coefficient, or log P, was calculated as

$$log P = \underline{⌈ dendrimer in octanol ⌉}$$

$$log dendrimer in PBS$$

The method was validated by comparing the tested value with the literature reported values of rhodamine and dendrimers.

The hydrophobicity of the materials used is quantified by log P. Note that the log P values increase with an increase in hydrophobicity of the materials. FIG. 4A shows that the log P values of the first three types of surface-modified G2 dendrimers are all negative (−0.9±0.2 for G2-RITC-NH₂,−1.0±0.0 for G2-RITC-Ac, and −1.3±0.4 for G2-RITC- COOH). After conjugation with OA, the partition coefficients of the dendrimer-OA conjugates changed from negative to positive (1.2±0.0 for G2-RITC-NH₂OA_{2.3} and 1.4±0.1 for G2-RITC-NH₂-OA_{2.7} (Giri et al., Environ. Sci. Technol. Pharm. Res. 28(9): 2246-2260, 2011). To investigate the relationship between the partition coefficient and skin permeation efficiency, the skin permeation efficiencies of the various G2 PAMAM dendrimers with differing log P values using the Franz diffusion cells were compared. Because the amounts of dendrimers that permeated across the full skin layers were relatively negligible (<3%), the amount of materials remaining in the donor chambers after 24 hours was recorded to estimate the amounts in the skin layers (FIG. 4B). Whereas a large portion (88.6%) of the hydrophilic G2-RITC-COOH was still dispersed in the donor solution, the OA-dendrimer conjugates tended to partition more into the skin. As more OA was conjugated, less material was detected in the donor solution (44.1 and 36.8% of the original amounts of G2-RITC-NH₂-OA_{2.3} and G2-RITC-NH₂-OA_{2.7}, respectively), which correlates well with the partition coefficient results and confirms that the partition coefficient is a good indicator for the skin-partitioning behavior of materials. The skin absorption of the rest of the conjugates was also calculated by measuring the amounts in the donor chambers: 62.7% for G2-RITC-NH₂ and 48.0% for G2-RITC-Ac.

The third hypothesis regarding the effect of hydrophobicity on skin permeation was tested by altering the 1-octanol-to-PBS partition coefficient (log P) of G2-RITC-NH₂ by conjugation with different amounts of OA. The reported partition coefficient for G2-NH₂ is −2.0, and that of OA is 7.3 at pH 74 which indicates that the G2 PAMAM dendrimer is hydrophilic, whereas OA is highly hydrophobic. Because the optimum range of log P for SC partitioning is 1-3, neither of them is easily SC-permeable when used separately. It was tested whether the covalent conjugation of these molecules could significantly change their partitioning behavior and potentially make their joint log P fall into the optimum range for best skin permeability. The results showed that by attaching OA, the log P values for the conjugate was reversed from negative to positive (FIG. 4A). The transition from negative to positive was dependent on the number of the hydrophobic molecules attached. Increasing the number of OA on the surface of G2 dendrimers increased the hydrophobicity of the final conjugates.

Hydrophobic modification of the dendrimers resulted in log P values (1.2 and 1.4 for G2-RITC-NH2-OA_{2.3} and G2-RITC NH2-OA_{2.7}, respectively) that theoretically permit the dendrimer conjugates to readily partition into the SC. In fact, the results from the Franz cell experiments showed significantly enhanced skin partitioning of the dendrimer-OA conjugates (FIG. 4B). The increase in % permeation into the receiver solutions was marginal; however, this can be attributed to the use of pure PBS in the receiver chambers as opposed to adding ethanol as reported by others.(Filipowicz et al., Intl. J. Pharma. 408: 152-156, 2011; Borowska et al., Int. J. Pharma. 398: 185-189, 2010). Further studies need to done to confirm the upper limit of the partition coefficient that dendrimer-OA conjugates can or should not exceed. Whether the value should be less than 3 for optimal skin permeation is not yet tested in our case. A linear relationship between the partition coefficient and skin permeability might exist, which could be the subject of our future investigations.

Collectively, the results highlighted in Examples 1-5 confirmed the three hypotheses noted above, and resulted in the following conclusions: (i) smaller dendrimers penetrate the skin better than larger ones, that is, the skin permeation and penetration depth of G2 are superior to those of G4; (ii) surface modifications of PAMAM dendrimers increase skin permeation efficiencies and dictate penetration pathways; and (iii) the G2-RITC-NH2-OA conjugates with log P values between 1 and 3, the reportedly optimal range for skin partitioning, result in enhanced skin deposition, compared with not only unmodified dendrimers but also all other G2-RITC conjugates used in this study. The results indicate that by adjusting the stoichiometry of the dendrimer—model drug conjugation, the partition coefficient can be manipulated, which serves well as a predictor of skin permeation of the material. After surface modification with charged moieties and adjustment of log P values, G2 PAMAM dendrimers could be further modified through conjugation with drug molecules, targeting moieties, and imaging probes to become multifunctional, programmed nanocarriers to achieve controlled therapeutic administration through the transdermal route.

### Example 6

### Characterization of G2 Dendrimers conjugated to Endoxifen

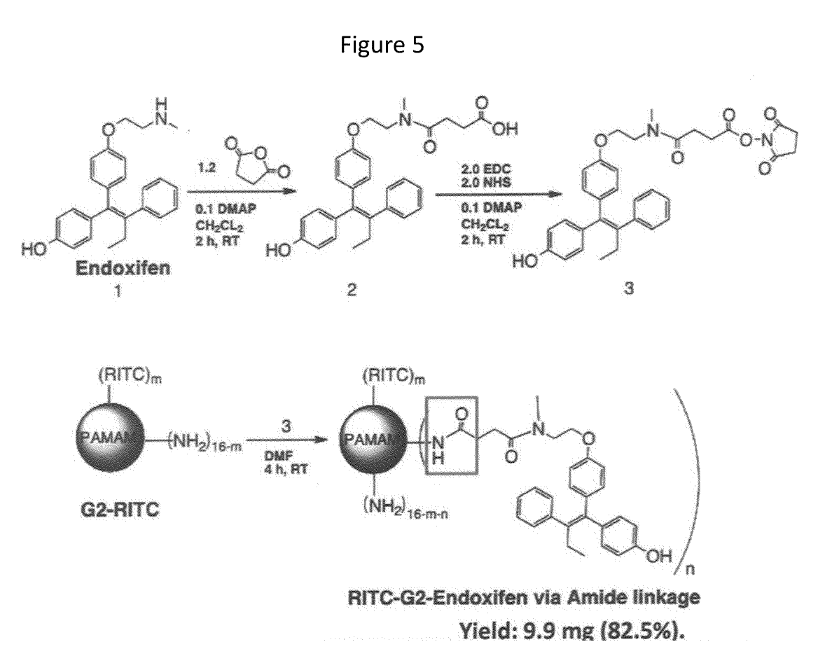

Endoxifen (ENX) was conjugated to G2-RITC using an amide linkage as described in Example 1 and depicted in FIG. 5. The yield was about 9.9 mg (82.5%) of the G2-RITC-ENX-NH₂.

UV and NMR characterization of the G2-RITC-ENX-NH₂ was carried out as described in Example 1. The G2-RITC-ENX-NH₂ were dissolved in 50% DMSO-50% water. The UV/vis measurements revealed that, on average, 1.25 RITC and 2.7 ENX were conjugated to each molecule, which are referred to as G2-RITC_{1.25}-ENX_{2.7}-NH₂. The RITC and ENX conjugation was confirmed using ¹H NMR by observing the peak(s) from the newly formed thiourea bond at 6.814 ppm as a result of conjugation between the dendrimers, RITC and ENX.

The skin penetration of endoxifen alone, G2-RITC-ENX-NH₂ and G2-RITC-NH₂ was measured using the Franz cell experiment as described in Example 2 every two hours (at 0, 2, 4, 6, 8, 10, 12, 14, 16, 18, 20, 22, 24 h), The experimental design is summarized in Table 3.

TABLE 3

| | | | | Vehicle control |
|---|---|---|---|---|
| Treatment | Endoxifen (ENX) | G2-RITC-ENX-NH₂ | G2-RITC-NH₂ | (water) |
| Repeats | n = 3 | n = 4 | n = 3 | n = 2 |
| Dosage | 108 μg/cell | 1 mM | 1 mM | 0 |
| | (0.5 mg/500 μL) | [2.5 mg/500uL] | [2.0 mg/500 μ1] | |
| | | (0.5 mg/500 μl EDX) | | |

The conjugation of ENX to PAMAM dendrimer greatly enhanced permeation through porcine skin. Figure X demonstrates the fold increases of the skin-absorbed materials during 24 h as measured by percent skin permeation into the receiver solution. G2-RITC-NH₂ exhibited skin permeation of 4.03% (±1.10) and G2-RITC-ENX-NH₂ exhibited skin permeation of 27.85% (±4.50) For this experiment, Flux1=22.523 μg/cm²/h, t_{tag}=6.47 h, permeability coefficient (Kp)=J/Cv=0.045 cm²/h, and diffusion coefficient (D)=h2/6tlag=0.7445*10-3 cm²/h.

Confirmation of dendrimer conjugate structure after skin permeation was investigated using fluorometry, high performance liquid chromatography (RP-HPLC) and mass-spectrometry. For fluorometry, EDX standards (20, 10, 5 μg/mL) at 390 nm after UV excitation for 10 minutes. excitation: 254 nm and emission scan at 300-500 nm. Ex: 254 nm, Em: 390 nm after UV lamp treatment for 10 minutes, RITC: Ex 555 nm, Em 590 nm w/o UV lamp excitation.

For RP-HPLC, endoxifen collected from the receiver solution was beyond detection limit (9 ng/mL). Recitation was x: 254 nm, Em: 390 nm after UV lamp treatment for 10 min. To identify G2-RITC-EDX conjugates from HPLC fractions mass-spectrometry was carried out. This allowed for an understanding of the degradation status and retention of the conjugates.

### Example 7

### Skin Penetration of Surface Modified G2 Dendrimers conjugated to RITC

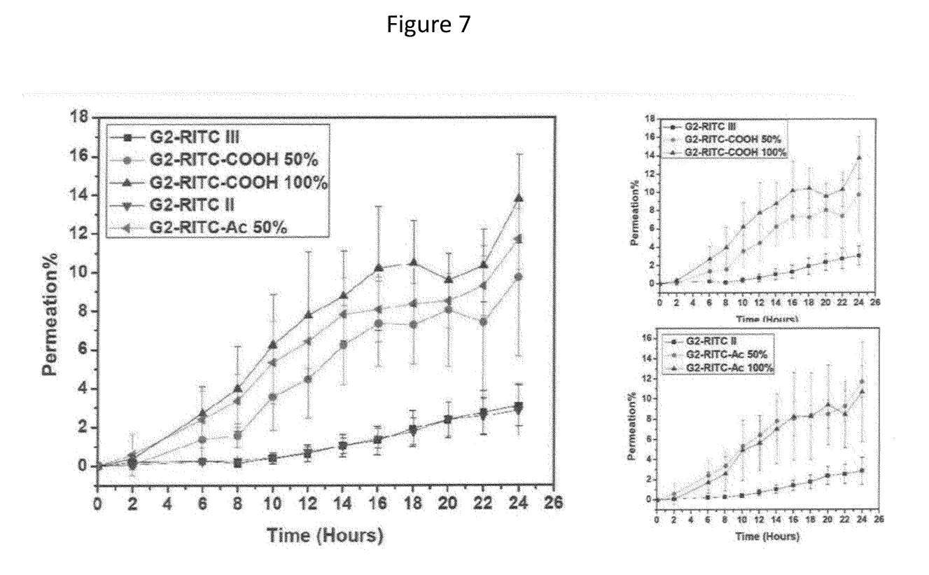

The surface of G2-RITC-NH₂ was modified by acetylation or carboxylation as described in Example 1 (See FIG. 2), The acetylation and carboxylation reactions were confirmed using NMR measurements MALD-TOF and titration. The surface charges (zeta potential, 169 millivolts) of the surface modified conjugates were determined and set out in Table 4. To assess the permeation efficiencies of the G2-RITC conjugates with different surface functional groups, Franz diffusion cell experiments were performed using a 1 m/vl concentration of the materials in ddH₂O without adding any commonly used permeation enhancers as described in Example 2. As shown in FIG. 7, there was an approximately up to 7-fold increase in permeation for both G2-RITC-COOH and G2-RITC-Ac compared with G2-RITC-NH2.

TABLE 4

| Surface Modification | Zeta potential | % Permeation |
|---|---|---|
| G2-RITC-NH₂—COOH 100% | −29.42 mV | 13.76 ± 2.31 |
| G2-RITC-NH₂—COOH 50% | −4.21 mV | 9.72 ± 4.09 |
| G2-RITC-NH₂—Ac 50% | 2.67 mV | 11.69 ± 1.58 |
| G2-RITC-NH₂—Ac 100% | 5.33 mV | 10.68 ± 4.96 |
| G2-RITC-NH₂ (batch I) | 11.83 mV | 2.84 ± 1.34 |
| G2-RITC-NH₂ (batch II) | 11.83 mV | 3.06 ± 1.05 |

1. A dendrimer conjugate comprising a surface-modified G1 to G5 poly(amidoamino) (PAMAM) dendrimer associated with a therapeutic agent, wherein the surface of the PAMAM dendrimer is modified to comprise at least 50% carboxylation or at least 50% acetylation and wherein the surface-modified PAMAM dendrimer increases skin penetration of therapeutic agent.

2. The dendrimer conjugate of claim 1 wherein the PAMAM dendrimer is selected from the group consisting of G1 PAMAM dendrimer, G2 PAMAM dendrimer, G3 PAMAM dendrimer, G4 PAMAM dendrimer and G5 PAM AM dendrimer.

3. A dendrimer conjugate of claim 1 wherein the PAMAM dendrimer comprises a chemical penetration enhancer (CPE).

4. The dendrimer conjugate of claim 3 wherein the CPE is selected from the group consisting of fatty acids, fatty alcohols, fatty acid esters, fatty alcohol ethers, biologics, enzymes, amines, amides, complexing agents, ionic compounds, dimetyl sulfoxide, N-methyl pyrrolidone, polar solvents, salicyclic acid, benzyl nicotinate, azones, polyhydric alcohols, oils, fatty ethers, urea, and surfactants or combinations thereof.

5. (canceled)

6. (canceled)

7. The dendrimer conjugate of claim 1 wherein the PAMAM dendrimer and therapeutic agent are in a physical mixture.

8. The dendrimer conjugate of claim 1 wherein the therapeutic agent is covalently associated to the PAMAM dendrimer.

9. The dendrimer conjugate of claim 1 wherein the therapeutic agent is associated to the PAMAM dendrimer by an amide bond.

10. The dendrimer conjugate of claim 1 wherein the therapeutic agent is selected from the group consisting of anticancer agents, chemopreventive agents, anesthetics, anorexics, anti-allergics, antiarthritics, antiasthmatic agents, antibiotics, anticholinergics, anticonvulsants, antidepressants, antihemophilics, antidiabetic agents, antidiarrheals, antifungals, antigens, antihistamines, antihypertensives, anti-inflammatories, antimigraine preparations, antinauseants, antineoplastics, antiparkinsonism drugs, antiprotozoans, antipruritics, antipsychotics, antipyretics, antispasmodics, antivirals, calcium channel blockers, cardiovascular preparations, central nervous system stimulants, contraceptives, cough and cold preparations including decongestants, diuretics, enzyme inhibitors, enzymes, genetic material including DNA and RNA, growth factors, growth hormones, hormone inhibitors, hypnotics, immunoactive agents, immunosuppressive agents, microbicides, muscle relaxants, parasympatholytics, peptides, peripheral and cerebral vasodilators, proteins, psychostimulants, receptor agonists, sedatives, spermicides and other contraceptives, steroids, sympathomimetics, tranquilizers, vaccines, vasodilating agents including general coronary, viral vectors and small organic molecules.

11. (canceled)

12. (canceled)

13. A composition comprising a dendrimer conjugate of claim 1 and a carrier.

14. The composition of claim 13 wherein the carrier is formulated for transdermal delivery.

15. The composition of claim 13 further comprising a chemical penetration enhancer (CPE).

16. The composition of claim 15 wherein the CPE is associated with the PAMAM dendrimer.

17. The composition of claim 15 wherein the CPE is selected from the group consisting of fatty acids, fatty alcohols, fatty acid esters, fatty alcohol ethers, biologics, enzymes, amines, amides, complexing agents, ionic compounds, dimetyl sulfoxide, N-methyl pyrrolidone, polar solvents, salicyclic acid, benzyl nicotinate, azones, polyhydric alcohols, oils, fatty ethers, urea, and surfactants or combinations thereof.

18. (canceled)

19. (canceled)

20. The composition of claim 13, wherein the carrier is a liquid, gel, solvent, liquid diluents, solubilizer, hydrogel, paraffin, wax, oil, silicone, ester, oily cream, aqueous cream, water soluble base, glycerol, glycol, lotion, polymer, powder or microemulsion.

21. The composition of claim 13 wherein the composition is attached to or within a device for transdermal delivery.

22. The composition of claim 21 wherein the device is a patch, gauze, adhesive bandage, pressure sensitive adhesive, microchip or microneedle.

23. A method of transdermal delivery of a therapeutic agent to a subject comprising contacting a surface modified G1 to G5 poly(amidoamino) (PAMAM) dendrimer associated with the therapeutic agent with the skin of the subject, wherein the surface of the PAMAM dendrimer is modified to comprise at least 50% carboxylation or at least 50% acetylation, wherein the surface modified PAMAM dendrimer increases penetration of the therapeutic agent into the skin of the subject.

24. The method of claim 26 herein the contacting of the dendrimer occurs at a site of need in the subject.

25. The method of claim 23 wherein transdermal delivery of the therapeutic agent decreases systemic exposure of the therapeutic agent in the subject.

26. (canceled)

27. (canceled)

28. A method of decreasing systemic exposure of a therapeutic agent in a subject comprising administering a surface modified G1 to G5 poly(amidoamino)(PAMAM) dendrimer associated with a therapeutically effective dose of a therapeutic agent on the skin of the subject at a site of need, wherein the surface of the PAMAM dendrimer is modified to comprise at least 50% carboxylation or at least 50% acetylation and wherein the surface modified PAMAM dendrimer increases skin penetration of the therapeutic agent and wherein the therapeutic agent is administered at a dose that is less than a therapeutically effective dose of the therapeutic agent administered orally or intravenously.

29-31. (canceled)
